(19) United States
(12) Patent Application Publication
(10) Pub. No.: US 2024/0081888 A1
(43) Pub. Date: Mar. 14, 2024

(54) RADIOFREQUENCY GENERATORS, SYSTEMS, AND METHODS

(71) Applicant: Stratus Medical, LLC, Magnolia, TX (US)

(72) Inventors: Bret Boudousquie, The Woodlands, TX (US); Eric Hyman, Wakefield, MA (US); Lucas Fichtel, Ashburnham, MA (US); Johnathan Goree, Little Rock, AR (US)

(21) Appl. No.: 18/222,994

(22) Filed: Jul. 17, 2023

Related U.S. Application Data
(60) Provisional application No. 63/389,521, filed on Jul. 15, 2022.

(51) Int. Cl. A61B 18/12; A61B 34/00

(52) U.S. Cl. CPC A61B 18/1206; A61B 34/25; A61B 2018/00577; A61B 2018/1253; A61B 2018/126

(57) ABSTRACT

In some instances, a generator includes a plurality of ports, each port able to individually couple with a separate radiofrequency probe; a touchscreen; and a control unit communicatively coupled with the touchscreen to cause the touchscreen to show a home display that includes a monopolar object and a bipolar object; operate the plurality of ports in a monopolar mode upon receiving a control signal from the touchscreen corresponding with actuation of the monopolar object or operate at least two of the plurality of ports as a coupled bipolar pair in a bipolar mode upon receiving a control signal from the touchscreen corresponding with actuation of the bipolar object; cause the touchscreen to continuously display a homing object while the generator is in either the monopolar mode or the bipolar mode; and upon actuation of the homing object, cause the touchscreen to show the home display again.

### CROSS-REFERENCE TO RELATED APPLICATIONS

This application claims priority benefit under 35 U.S.C. § 119(e) of U.S. Provisional Patent Application No. 63/389,521, filed Jul. 15, 2022, titled RADIOFREQUENCY GENERATORS, SYSTEMS, AND METHODS, the entire contents of which are hereby incorporated by reference herein.

### TECHNICAL FIELD

Certain embodiments described herein relate generally to generators and related systems and methods, and further embodiments relate more particularly to such generators, systems, and methods suitable for used in radiofrequency (RF) procedures, such as RF ablation and/or pulsed RF procedures.

### BACKGROUND

Radiofrequency generators are used in a variety of medical procedures. In some instances, the radiofrequency generators may be used in radiofrequency ablation procedures, such as, for example, RF ablation and/or pulsed RF procedures for the treatment of pain. In some instances, needles are inserted into a patient to a target site and probes are inserted into the needles to energize the probes. The probes may be coupled to and powered by a radiofrequency generator. In various instances, the generator may be capable of operating the probes in a monopolar mode, such as by used with a return electrode positioned on the skin of a patient, a bipolar mode, in which energy is transmitted between, e.g., adjacent needles, and a pulsed mode, in which pulses of RF energy are delivered. Various known generators suffer from a variety of drawbacks, however. Embodiments disclosed herein remedy, ameliorate, or avoid one or more of these drawbacks and/or can be advantageously time saving and/or can be readily implemented and/or understood by a user.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is an elevation view, with certain components depicted schematically, of an embodiment of a system that includes a generator that includes multiple ports and multiple instrument assemblies that can be coupled interchangeably with any of the ports of the generator;

FIG. 2 is an elevation view of an embodiment of a radiofrequency probe that includes an embodiment of a connector that is couplable with any of the ports of the control unit of FIG. 1;

FIG. 3A is a schematic diagram depicting various communicative and power couplings among components of the system of FIG. 1;

FIG. 3B is a more detailed schematic diagram depicting various communicative and power couplings among components of a generator compatible with the system of FIG. 1;

FIG. 4 depicts a home screen that is displayable on the generator of the system depicted in FIG. 1, the home screen being readily accessible from other screens that are separately displayable on generator, the screen further providing access to any of a plurality of predetermined operational modes of the generator in one or more steps in a variety of illustrative RF procedures;

FIG. 5A depicts an illustrative monopolar settings screen that, in some embodiments, corresponds with a monopolar mode of operation of the generator and, in further embodiments, is directly accessible from the home screen of FIG. 4.

FIG. 5B depicts the monopolar settings screen in a further operational state in which sensory and motor stimulation buttons have been selected;

FIG. 5C depicts an embodiment of a popup screen that facilitates entry of settings for an illustrative monopolar procedure;

FIG. 5D depicts an illustrative monopolar sensory stimulation screen;

FIG. 5E depicts an illustrative monopolar motor stimulation screen;

FIG. 5F depicts an illustrative monopolar lesion screen;

FIG. 5G depicts the monopolar lesion screen in a further operational state in which temperature and lesion time settings can be adjusted;

FIG. 5H depicts the monopolar lesion screen in a further operational state in which a user is prompted to confirm a command to start all monopolar channels;

FIG. 5I depicts the monopolar lesion screen in a further operational state in which three separate channels are being energized simultaneously to form lesions within a patient;

FIG. 5J depicts an embodiment of a popup screen that provides a more detailed view of graphed data for an ongoing monopolar lesion event;

FIG. 5K depicts the monopolar lesion screen in a further operational state in which the lesioning events of three separate channels are complete;

FIG. 5L depicts a monopolar procedure summary screen;

FIG. 5M depicts an embodiment of a popup screen that provides a more detailed view of graphed data for a completed monopolar lesion event;

FIG. 5N depicts an alternative embodiment of a monopolar sensory stimulation screen;

FIG. 6A depicts an illustrative bipolar settings screen that, in some embodiments, corresponds with a bipolar mode of operation of the generator and, in further embodiments, is directly accessible from the home screen of FIG. 4.

FIG. 6B depicts the bipolar settings screen in another operational state in which pairings of four channels have been reassigned;

FIG. 6C depicts the bipolar settings screen in a further operational state in which the pairings of the four channels have been reassigned again to the same configuration as that depicted in FIG. 6A and in which sensory and motor stimulation buttons have been selected;

FIG. 6D depicts an embodiment of a popup screen that facilitates entry of settings for an illustrative bipolar procedure;

FIG. 6E depicts an illustrative bipolar sensory stimulation screen;

FIG. 6F depicts the bipolar sensory stimulation screen in a further operational state in which a sensory stimulation event is ongoing;

FIG. 6G depicts an illustrative bipolar motor stimulation screen;

FIG. 6H depicts an illustrative bipolar lesion screen;

FIG. 6I depicts the monopolar lesion screen in a further operational state in which a lesioning event of first channel pairing has been paused and a lesioning event of a second channel pairing is ongoing;

FIG. 6J depicts an embodiment of a popup screen that provides a more detailed view of graphed data for an ongoing bipolar lesion event;

FIG. 6K depicts the bipolar lesion screen in a further operational state in which the lesioning events of two separate channel pairings are complete;

FIG. 6L depicts a bipolar procedure summary screen;

FIG. 6M depicts an embodiment of a popup screen that provides a more detailed view of graphed data for a completed bipolar lesion event;

FIG. 6N depicts an alternative embodiment of a bipolar sensory stimulation screen;

FIG. 7A depicts an illustrative combination settings screen that, in some embodiments, corresponds with a combination mode of operation of the generator in which at least one channel operates in a monopolar mode and a pairing of channels operates in a bipolar mode, the combination settings screen, in further embodiments, being directly accessible from the home screen of FIG. 4.

FIG. 7B depicts the combination settings screen in another operational state in which a pairing of channels has been established;

FIG. 7C depicts the combination settings screen in another operational state in which four channels are available from which to establish a bipolar pairing;

FIG. 7D depicts the combination settings screen in another operational state in which two of the available channels of FIG. 7C have been paired for bipolar operation;

FIG. 7E depicts an embodiment of a popup screen that facilitates entry of settings for an illustrative combination procedure, with settings for each of the monopolar and bipolar portions of the procedure being adjustable;

FIG. 7F depicts an illustrative combination sensory stimulation screen;

FIG. 7G depicts an illustrative combination motor stimulation screen;

FIG. 7H depicts an illustrative combination lesion screen;

FIG. 7I depicts the combination lesion screen in another operational state and/or in a different procedure in which a different bipolar pairing of channels has been selected, a lesioning event of a first monopolar channel has been paused, and lesioning events of a bipolar pairing of channels and second monopolar channel are ongoing;

FIG. 7J depicts an embodiment of a popup screen that provides a more detailed view of graphed data for an ongoing monopolar portion of a combination lesion event;

FIG. 7K depicts the combination lesion screen in a further operational state in which the lesioning events of the separate monopolar channels and the bipolar pairing are complete;

FIG. 7L depicts a combination procedure summary screen;

FIG. 8A depicts an illustrative pulsed monopolar settings screen that, in some embodiments, corresponds with a pulsed monopolar mode of operation of the generator and, in further embodiments, is directly accessible from the home screen of FIG. 4;

FIG. 8B depicts an embodiment of a popup screen that facilitates entry of settings for an illustrative timed pulsed monopolar procedure;

FIG. 8C depicts an embodiment of a popup screen that facilitates entry of settings for an illustrative dosed pulsed monopolar procedure;

FIG. 9 depicts an illustrative saved procedures screen that, in some embodiments, permits creation of and/or access to end-user customizable versions of various modes of operation of the generator and, in further embodiments, is directly accessible from the home screen of FIG. 4;

FIG. 10 depicts an illustrative system settings screen that, in some embodiments, permits creation of and/or access to end-user customizable versions of various modes of operation of the generator and, in further embodiments, is directly accessible from the home screen of FIG. 4;

FIG. 11A depicts an illustrative monopolar advanced settings screen via which selections and adjustments can be made to various parameters of sensory and motor stimulation sub-procedures;

FIG. 11B depicts the monopolar advanced settings screen of FIG. 11A with the motor and sensory sections of the display shown in reversed order by virtue of actuation of an object positioned between these sections on the display;

FIG. 12 depicts an illustrative pulsed bipolar settings screen, which can be accessed through one or more indirect pathways from the home screen of FIG. 4, or stated otherwise, which in some embodiments is not directly accessible from the home screen of FIG. 4;

FIG. 13A depicts a flow chart showing interrelations among steps in numerous illustrative methods that may be implemented via the system of FIG. 1 and/or the generator thereof;

FIG. 13B depicts another flow chart showing interrelations among steps in numerous illustrative methods that may be implemented via the system of FIG. 1 and/or the generator thereof, wherein the sensory stimulation and motor stimulation steps appear in reverse order from those depicted in FIG. 13A, which, in some embodiments, can be accomplished via actuation of the object depicted in FIG. 11B as mentioned above; and

FIG. 13C depicts another flow chart showing interrelations among steps in numerous illustrative methods that may be implemented via the system of FIG. 1 and/or the generator thereof, wherein a home screen associated with the generator includes an actuatable object for directly accessing a pulsed bipolar procedure.

### DETAILED DESCRIPTION

Embodiments of the present disclosure relate generally to generators, systems, and methods for radiofrequency (RF) procedures. In certain embodiments, the generator is simultaneously couplable with multiple instruments, such as RF probes, which may be inserted into respective needles for RF procedures, such as RF ablation. For example, in some instances, each of the RF probes is associated with a separate cable that can be coupled with the generator, such as via a separate port, and is inserted in a separate needle that is inserted into a patient. Thus, for example, the generator can include multiple ports, each port being couplable with a separate RF probe. In some instances, each of the ports may be operable with any of the RF probes. Stated otherwise, the RF probes may be coupled interchangeably with any of the ports of the generator.

In various embodiments, the RF probes and needles may be operated in a monopolar mode, a bipolar mode, or a combined or combination mode (in which one or more of the RF probes and needles are operated in a monopolar mode and a pair of RF probes and needles are operated in a bipolar mode). For convenience, the present disclosure may at times refer only to control or energization of the RF probes by the generator; it is understood that the RF probes may be positioned within needles via which RF energy is delivered to a patient during such control or energization.

While certain known generators may be capable of use in each of a monopolar mode, a bipolar mode, or a combination mode, it may be time consuming and/or non-intuitive for practitioners, lab technicians, and/or other users of the generators to set up or otherwise configure the generators for use in each such mode. Embodiments herein provide an intuitive interface and/or controlled environment in which a user is guided or stepped through each of a variety of operational modes of a generator, including, e.g., two or more of monopolar, bipolar, combined, and pulsed modes, to properly utilize probes connected to the generator. In further instances, a user can readily select a saved procedure, which may be any of a monopolar, bipolar, combined, or pulsed mode. In various instances, the pulsed modes may include monopolar and/or bipolar configurations.

Other or further embodiments described herein include a touchscreen via which a series of visual displays, which may also be referred to herein as screens, are provided to a user in manners that facilitate selection of a desired procedure, facilitates conduction of the desired procedure, and/or assists the user in properly conducting the desired procedure. In some instances, a home screen visual display, which may also be referred to herein as a home display, is provided as a gateway to exclusively entering any one of several available operation modes of the generator, such as a monopolar, bipolar, or combination mode of operation. Upon selection of a desired operational mode, the generator can display to the user a series of visual displays that aids the user in configuring the generator for a desired procedure. The generator can step the user through sequential setup events and/or stimulation events (e.g., sensory stimulation and/or motor stimulation) before ultimately providing the user with a visual display from which the user can elect to energize the probes to commence an ablation or other RF treatment.

Other or further embodiments allow for ready assignment or reassignment of probe pairings in the bipolar mode of operation. For example, in some embodiments, the generator can be configured to control two probes in a first bipolar pairing and/or two additional probes in a second bipolar pairing. For example, the generator may control four RF probes in two separate bipolar pairings sequentially and/or simultaneously. In some embodiments, when all four probes are connected to the generator, the generator automatically pairs two sets of the probes when in the bipolar mode. For example, in embodiments of a system that includes probes “a,” “b,” “c,” and “d,” probes “a” and “b” may be paired automatically and probes “c” and “d” likewise may be paired automatically. The generator further permits a user to readily re-pair or elicit an alternate coupling of the probes such that each probe of the first set is unpaired from the other probe of the first set, each probe of the second set is unpaired from the other probe of the second set, and one probe from the first set is paired with one probe of the second set and the other probe from the first set is paired with the other probe of the second set. For example, in embodiments of the system that includes probes “a,” “b,” “c,” and “d,” probe “a” is disassociated from probe “b” and newly paired with probe “d,” and probe “b” is disassociated from probe “a” and newly paired with probe “c.” In some embodiments, the re-pairing may be achieved with the press of a single button (e.g., a dedicated button, a soft button, or virtual button on a touchscreen).

In some embodiments, in stepping a user through a procedure (e.g., monopolar or bipolar), the generator may default to performing sensory stimulation prior to motor stimulation in advance of performing an ablation procedure, where both sensory and motor stimulation options have been selected. In some instances, a user may instead desire to perform motor stimulation prior to sensory stimulation, where both procedures are desired. Certain embodiments permit a user to readily revers the order in which the sensory and motor stimulation procedures are performed.

The foregoing and/or other advantages of various embodiments will be apparent from the disclosure that follows.

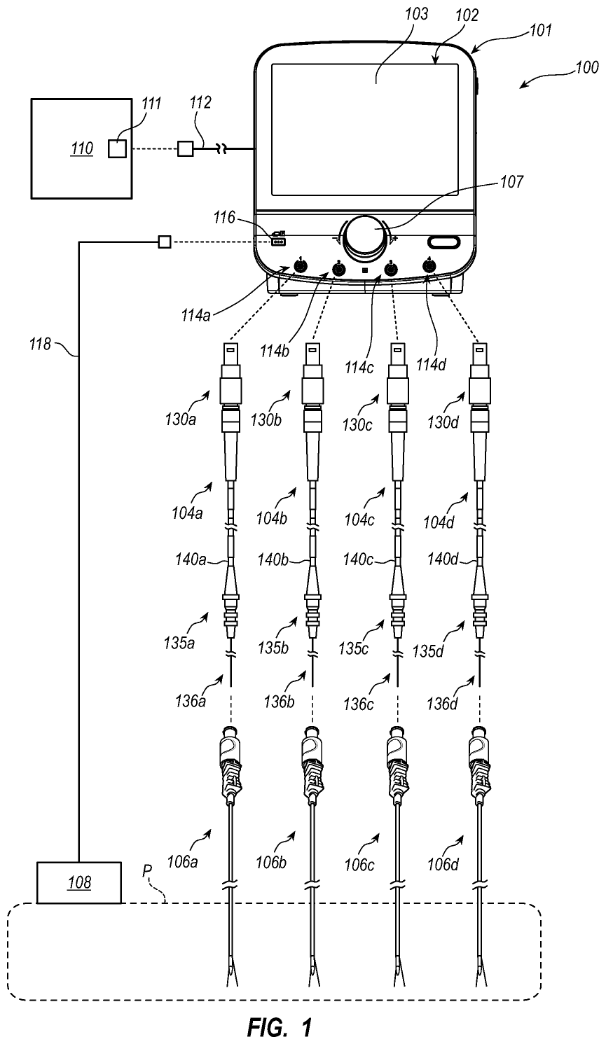

FIG. 1 depicts an embodiment of a system 100 that includes a generator 101 and a plurality of instrument assemblies 104a-d that are operable or controllable by the generator 101. In various embodiments, the illustrated system 100 can be described as a medical system, an RF treatment system, and/or an RF ablation system. In some embodiments, the system 100 is particularly well-adapted for use in RF neurotomy, and may be referred to as an RF neurotomy system.

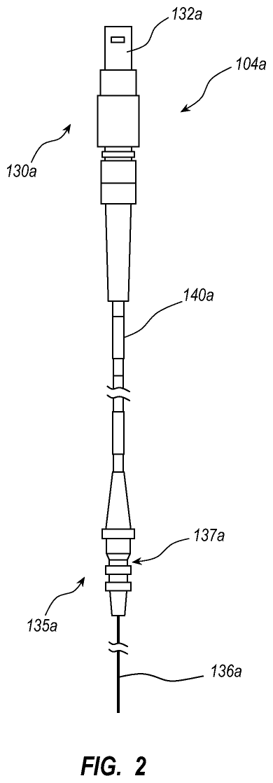

Each of the instrument assemblies 104a-d may also or alternatively be referred to as a medical instrument assembly or as an RF probe assembly. Each assembly 104a-d includes an RF probe 135a-d coupled to a distal end of a cable 140a-d, and a connector 130a-d is coupled to a proximal end of the cable 140a-d. Each RF probe 135a-d includes an active element or electrode 136a-d of the RF probe 135a-d (while in some instances, the electrode 136a-d may itself be referred to as a probe). As shown in FIG. 2, the RF probe 135a (representative of the additional probes 135b-d) may include a hub 137a to which each of the electrode 136a and the cable 140a are attached. In various embodiments, one or more of the instrument assemblies 104a-d can be a NIMBUS® Probe, available from STRATUS® Medical, LLC, of Magnolia, Texas.

With further reference to FIG. 1, in some embodiments, each of the instrument assemblies 104a-d may be usable with a further medical instrument 106a-d, which can be couplable with the RF probes 135a-d and can be considered as selectively attachable and/or selectively removable components of the respective instrument assemblies 104a-d. For example, in the illustrated embodiment, each medical instrument 106a-d comprises an RF neurotomy needle that is configured to receive therein the electrode 136a-d of a respective RF probe 135a-d. In various embodiments, the electrodes 136a-d can energize a needle tip, or a needle tip and/or deployable tines, of the medical instrument 106a-d. For example, in various embodiments, one or more of the medical instruments 106a-d can be or resemble any of the embodiments of RF needles described in U.S. Pat. No. 10,716,618, titled SYSTEMS AND METHODS FOR TISSUE ABLATION, issued Jul. 21, 2020, and/or U.S. Pat. No. 10,736,688, titled METHODS AND SYSTEMS FOR RADIO FREQUENCY NEUROTOMY, issued Aug. 11, 2020, the entire contents of each of which are hereby incorporated by reference herein. In various embodiments, one or more of the medical instruments 106a-d can be a NIMBUS® Multi-tined Expandable Electrode, available from STRATUS® Medical, LLC, of Magnolia, Texas. In other or further embodiments, one or more of the medical instruments 106a-d can be a VESTA® RF Cannula, available from Stratus™ Medical, LLC, of Magnolia, Texas.

In some embodiments, the generator 101 can operate one or more of the medical instruments 106a-d in a monopolar fashion or mode. For example, in some embodiments, a grounding or electrode pad 108 may be affixed to the skin of the patient P, and an electrical cable 118 extending from the grounding pad 108 can be coupled with a grounding pad port 116 of the generator 101. For each of the one or more medical instruments 106a-d that will be operated in the monopolar mode, the connector 130a-d of the associated instrument assembly 104a-d can be coupled with the generator 101. The one more medical instruments 106a-d can be inserted into the patient P (e.g., into proximity to one or more nerves targeted for ablation). Where applicable or desirable, tines of the medical instruments 106a-d can be deployed. At any suitable stage, such as before or after insertion of one or more of the medical instruments 106a-d or before or after deployment of the tines of a given medical instrument 106a-d, the medical instrument 106a-d can be coupled with its respective RF probe 135a-d. For each medical instrument 106a-d operated in the monopolar mode, an electrical circuit can include, using just “a”-valued components as an example, the generator 101, the instrument assembly 104a, the medical instrument 106a, the patient P, the electrode pad 108, and the cable 118 back to the generator 101. Each of the “b”-, “c”-, and “d”-valued components can form similar individual monopolar circuits.

In some embodiments, the generator 101 can operate two or more of the medical instruments 106a-d in a bipolar fashion. For example, in some embodiments, the generator 101 can operate pairs of the medical instruments 106a-d (e.g., the medical instruments 106a,b) in bipolar fashion, in which at any given instant of operation in the bipolar mode, one of the medical instruments 106a-d in a bipolar pair (e.g., the medical instrument 106a) serves as an active electrode, and the other medical instrument 106a-d in the bipolar pair (e.g., the medical instrument 106b) serves as a return electrode. The medical instruments 106a-d can be energized by the respective RF probes 135a-d inserted therein.

In some illustrative examples, the pairs of medical instruments 106a,b and/or 106c,d may be operated in a bipolar mode, whether separately (e.g., the pair of medical instruments 106a,b followed by the pair of medical instruments 106c,d) or simultaneously (e.g., the pair of medical instruments 106a,b at the same time as the pair of medical instruments 106c,d), depending on positioning of the medical instruments 106a-d within the patient P. In other or further illustrative examples, a different, alternative, reassigned, or, e.g., secondary pairing of the medical instruments 106a-d, such as the pair of medical instruments 106b,c and/or the pair of medical instruments 106a,d, may be operated separately or simultaneously, depending on positioning of the medical instruments 106a-d within the patient P. For each bipolar pairing of medical instruments 106a-d, an electrical circuit (using just “a”- and “b”-valued components as an example) can include the generator 101, the instrument assembly 104a, the medical instrument 106a, the patient P, the medical instrument 106b, and the instrument assembly 104b back to the generator 101. The “c”- and “d”-valued components, the “b”- and “c”-valued components, and the “a”- and “d”-valued components can form similar paired bipolar circuits.

As further discussed below, in various embodiments, certain bipolar pairings may be automatically assigned by the generator 101. In other or further embodiments, the bipolar pairings may be disassociated and new bipolar pairings readily achieved by the user, such as by the press of a single button (e.g., a dedicated button, a soft key, or a virtual button or virtual key on a touchscreen). In certain embodiments, a practitioner can implement the re-pairing without repositioning any of the needles 106a-b and/or without repositioning any of the RF probes 135a-d. Thus, for example, in initially placing the needles 106a-d, the practitioner might ensure that the needles 106a, 106b are sufficiently close to each other for bipolar operation therebetween and that the needles 106c, 106d are sufficiently close to each other for bipolar operation therebetween upon initial placement thereof. In other or further instances, the practitioner might instead or further ensure that the needles 106a, 106d are sufficiently close to each other for bipolar operation therebetween and that the needles 106b, 106c are sufficiently close to each other for bipolar operation therebetween upon said initial placement thereof. During a bipolar procedure using the generator 101, the probes 135a, b and 135 c, d, respectively (which are received in the needles 106a, b and 106c, d, respectively), may be automatically paired by the generator 101. The user may elect to conduct one or more bipolar ablations via these pairings, whether sequentially or simultaneously. The user may alternatively or further elect to conduct one or more bipolar ablations via the reassigned pairing of the RF probes 135c, b and 135a, d, such as described above. In certain instances, the user may proceed with the one or more bipolar ablations (e.g., originally or in addition to previous ablations) in the reassigned pairing without first repositioning any of the needles 106a-d and/or any of the RF probes 135a-d.

In some instances, the user may only elect to conduct bipolar ablations between pairings of RF probes 135 and associated needles 106 that are sufficiently close to each other to permit such bipolar operation. In some instances, only one set of paired RF probes/needles 106 may be used to create an ablation during the initial and/or reassigned pairing described in the previous paragraphs.

In some instances, a practitioner may position each of the medical instruments 106a-d at different elevations of a spine of the patient P in the order shown. The practitioner may select, via the generator 101, a bipolar pairing of the medical instruments 106a,b and a further bipolar pairing of the medical instruments 106c,d for a first ablation procedure. For example, RF energy may be applied simultaneously through each bipolar pairing during a first “burn” or ablatio nevent, thereby forming a lesion within the patient P that extends at least between the electrodes of the medical instruments 106a,b and another lesion within the patient P that extends at least between the electrodes of the medical instruments 106c,d. Subsequently, a practitioner may select, via the generator 101, a bipolar pairing of the medical instruments 106b,c for a second ablation procedure, which can proceed without changing the position of at least the medical instruments 106b,c or their associated instrument assemblies 104b,c. The practitioner may then proceed with a second “burn” or ablation event, in which RF energy is applied through the circuit that includes the medical instruments 106b,c, thereby forming a lesion within the patient P that extends at least between the electrodes of the medical instruments 106b,c.

In still other or further embodiments, the “a”- and “c”-valued components and/or the “b”- and “d”-valued components can form paired bipolar circuits, which may be selectable by a user via the generator 101. As previously noted, in some instances, an appropriateness of such pairings may depend upon relative positioning of the various medical instruments 106a-d within the patient P.

With continued reference to FIG. 1, the generator 101 can be of any suitable variety. For example, in the illustrated embodiment, the generator 101 includes an RF generator. In some embodiments, the RF generator is configured to operate one or more RF probe/needle assemblies in a monopolar mode and/or two or more RF probe/needle assemblies in a bipolar mode, such as in manners described above. In further embodiments, the RF generator may be configured to operate one or more of the probe/needle assemblies in a monopolar mode and a pairing of the probe/needle assemblies in a bipolar mode, which may be referred to as a combined mode or a combination mode of operation. In other or further embodiments, the generator may be configured to operate one or more of the probe/needle assemblies in a pulsed RF mode, such as by applying electrical pulses through one or more of the RF probes 135a-d in a monopolar configuration and/or by applying electrical pulses through any suitable pairing of the RF probes 135a-d in a bipolar configuration.

In certain embodiments, the generator includes a display unit 102, which may include a monitor, display, or screen 103. In some embodiments, the display unit 102 may also be used as an input device. For example, in some embodiments, the screen 103 is a touchscreen that can be manipulated by a user to interface with the generator 101, as discussed further below. Any suitable screen 103, such as any suitable touchscreen, is contemplated. The screen 103 may alternatively be referred to herein as the touchscreen 103.

In the illustrated embodiment, any suitable power source 110 can be coupled with the generator 101. In the illustrated embodiment, the power source 110 comprises dedicated electrical wiring and an electrical outlet 111, to which can be coupled a power cord 112 of the generator 101.

With continued reference to FIG. 1, the generator 101 can include a plurality of ports 114a-d that can each be coupled individually with one of the instrument assemblies 104a-b. In some embodiments, the ports 114a-d can be used interchangeably, or stated otherwise, each of the instrument assemblies 104a-b can be coupled interchangeably with any of the ports 114a-d. For example, in some embodiments, a single instrument assembly 104a can be used with any of the ports 114a-d. In certain of such embodiments, the generator 101 can operate the associated medical instrument 106a-d (e.g., the medical instrument 106a) in a monopolar mode equally well from any of the ports 114a-d. As a further example, in some embodiments, two instrument assemblies 104a-d (e.g., the instrument assemblies 104a,b) can be used with any two of the ports 114a-d. In certain of such embodiments, the generator 101 can operate the associated medical instruments (e.g., the RF needles 106a,b) either individually, in a monopolar mode, or as a pair, in a bipolar mode, equally well from any pairing of the ports 114a-d.

As shown in FIG. 1, in some embodiments, the generator 101 can include other input devices by which a user may communicate commands to the generator. For example, in addition to the touchscreen 103, the generator 101 includes a turn dial 107. The turn dial 107 may, for example, provide control signals via any suitable rotary encoder. Any other suitable electromechanical, optomechanical, or other suitable device that may be coupled with the physically manipulable knob or dial 107 is contemplated. In still other or further embodiments, one or more alternative or additional input devices may be used, including one or more of a mouse, keyboard, app on a personal electronic device (e.g., phone or tablet), etc.

In certain embodiments, the touchscreen 103 need not be physically incorporated into the generator 101 device itself. For example, in some embodiments, the touchscreen 103 may be separate from the device and in wired or wireless communication therewith. In some embodiments, the touchscreen 103 can include the touchscreen of a separate personal electronic device, such as a phone or tablet, that is configured to communicate with the generator 101 in any suitable manner, such as, for example, wirelessly (e.g., via Bluetooth, WiFi, or any other suitable protocol).

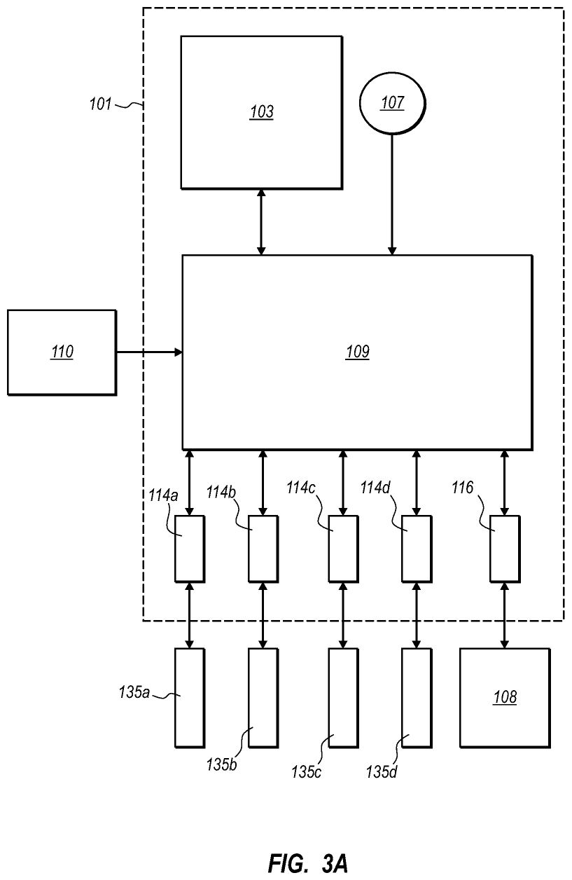

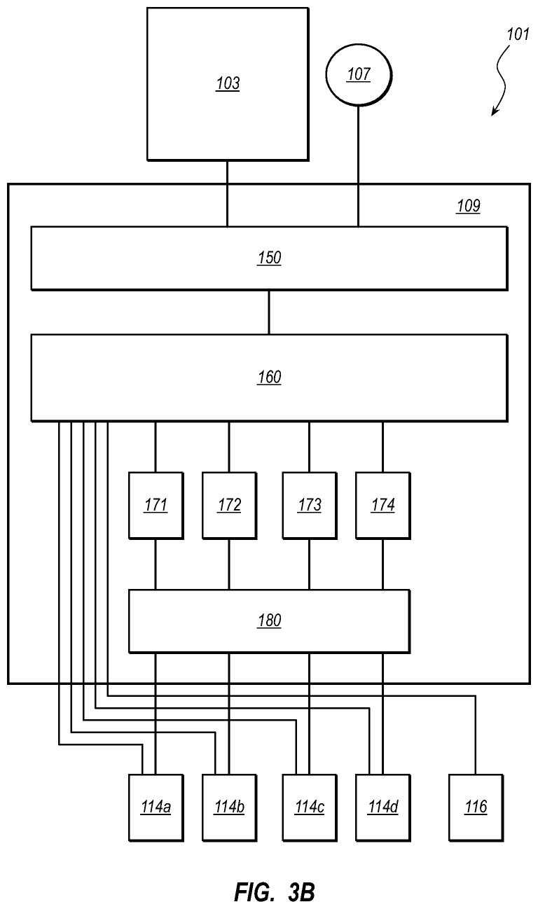

With reference to FIG. 3A, the generator 101 can include a control unit 109 that includes any suitable electronic architecture configured to control any of the probes 135a-d when the probes 135a-d are coupled with respective ports 114a-d of the generator 101. For example, in some embodiments, the control unit 109 can include one or more preprogrammed microcontrollers and/or field programmable gate arrays (FPGAs) that include machine-executable instructions stored in nonvolatile memory for controlling the one or more probes 135a-d via their connections with the ports 114a-d. Any other suitable electronic control components are contemplated. In certain embodiments, the probes 135a-d may include sensors, such as thermocouples, and temperature information obtained thereby may be communicated to the control unit 109 via the ports 114a-d.

The control unit 109 can further include any suitable electronic architecture for appropriately distributing power received from the power source 110 to the probes 135a-d via the ports 114a-d. Power delivery may be controlled, at least in part, via the microcontrollers and/or FPGAs. The power may be delivered to the probes 135a-d as radiofrequency signals or pulses in any suitable manner.

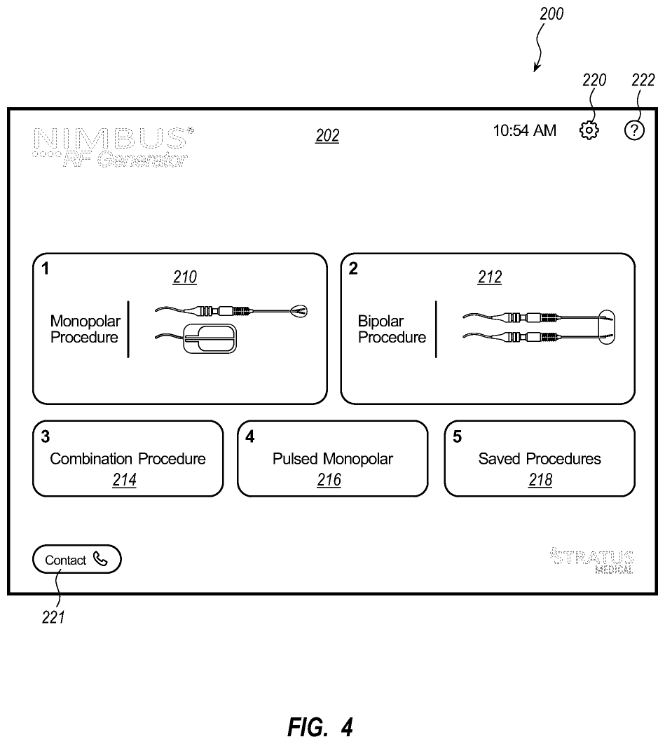

As previously noted, each of the touchscreen 103, the turn dial 107, and/or any other suitable input devices can be communicatively coupled with the control unit 109 so as to communicate instructions received from a user to the control unit 109. The control unit 109 may further be communicatively coupled with the touchscreen 103 so as to provide, control, and/or cause to be shown on the touchscreen 103 visual displays. The visual displays, which are shown on the touchscreen 103, e.g., individually, in selected succession, and/or in predetermined sequential manners, may also or alternatively be referred to as a graphical user interface of the generator 101. For example, in various embodiments, the control unit 109 can change the visual displays, e.g., based at least in part on instructions received from a user, to any or all of those depicted in FIGS. 4 through 12. A user may receive information from and/or provide instructions to the control unit 109 by touching, pressing, sliding, or otherwise suitably manipulating one or more actuatable objects, such as virtual buttons or soft keys, present in the visual displays.

FIG. 3B depicts a more detailed schematic view of an illustrative embodiment of the generator 101. As previously noted, the touchscreen 103 and the knob or dial 107 can be communicatively coupled with the control unit 109. In some embodiments, the control unit 109 can receive input (e.g., instructions or commands) from each of the touchscreen 103 and the dial 107. In further instances, the control unit 109 can provide instructions or commands to the touchscreen 103 or otherwise cause the touchscreen 103 to show various displays, which may permit interaction with a user, as discussed further below.

In certain embodiments, the control unit 109 includes a controller 150, which can include any suitable variety of one or more controllers, microcontrollers, processors, or the like. The controller 150 can include one or more processors configured to control the graphical user interface, an operating system, and ablation and stimulation waveforms. The controller 150 may control the therapy delivered to the patient, monitor system status, calculate or otherwise process therapy data, and report therapy status information to touchscreen 103 to present to the user. In some embodiments, the controller 150 can provide proportional-integral-derivative (PID) controls of RF channels 171, 172, 173, 174. For example, the controller 150 may be configured to communicate with channel control circuitry 160, which in some embodiments may include a field-programmable gate array (FPGA). The controller 150 can control certain operations of the FPGA 160, which in turn can control aspects of the RF channels 171, 172, 173, 174. The controller 150 may configure RF control interfaces and/or multiplexer (Mux) interfaces of the channel control circuitry 160. In some embodiments, the controller 150 may gather FPGA RF measurements and/or probe temperature measurements from the channel control circuitry 160.

The controller 150 can include a processor that provides a user interface for the touchscreen 103. The controller 150 may receive user inputs from the touchscreen 103.

As previously noted with respect to FIG. 3A, the control unit 109 can receive power from an external power source 110. In certain embodiments, at least some portion of the power is provided to each of the controller 150 and the FPGA 160 and can be managed and distributed in any suitable manner.

In certain embodiments, the channel control circuitry 160 may provide interfaces and a state machine to control the RF Mux 180. The RF Mux 180 may provide a configurable interface between the RF signals output by the RF channels 171, 172, 173, 174 and the ports 114a-d. For example, the channel control circuitry 160 may configure a state machine to control the RF Mux 180 to place the RF channels 171, 172, 173, 174 in monopolar or bipolar mode. The channel control circuitry 160 may provide interfaces and the state machine to read probe thermocouple temperature measurements.

Other control circuitry is contemplated. For example, in some embodiments, a single RF generation unit may be used and the RF Mux 180 can include and/or be replaced with a series of switches to control delivery of RF energy to each of the ports 114a-d in any suitable manner.

As discussed further below, the various visual displays may be provided to a user in a predetermined and/or sequential order. In some instances, each visual display may restrict the number of operations or selections a user may make therein or thereon. The visual displays may step the user through the setting up and/or executing or performing one or more varieties of RF treatment procedures (e.g., monopolar, bipolar, combination, pulsed), which may assist a user in properly proceeding through the one or more procedures. Stated otherwise, the sequential visual displays may guide a user through a series of focused or restricted environments to help ensure that appropriate operational parameters are entered in a stepwise and/or logical order for a given procedure. The screens or displays may be provided in a manner that is intuitive for those who are experienced with RF procedures and/or may help train and/or instruct those who are less familiar with RF procedures. In some instances, a home screen or home display can serve as a launching pad from which any of a variety of procedures can be selected. Once a particular procedure is selected, the user may then be guided through setting selections and sub-procedures (e.g., sensory stimulation and/or motor stimulation) that only pertain to the selected procedure. Once within the confines of the selected procedure, the user may be required to return to the home display in order to instead proceed with a different type of procedure. In some instances, an actuatable homing object that, when actuated, can return the user to the home display, may be present throughout (e.g., continuously throughout) any selected procedure to permit the user to readily return to the home display.

For example, a user may select a monopolar procedure from the home display. After advancing through the monopolar procedure, whether in part or in its entirety, the user may actuate the homing object to return to the home display. The user may then select a further monopolar procedure or a different procedure, such as a bipolar or combination procedure.

FIG. 4 depicts a visual display 200 that represents a home screen 202, which may also be referred to herein as a home display 202. The home screen 202 includes a plurality of actuatable objects 210, 212, 214, 216, 218 that a user can manipulate to select a desired mode of operation of the generator 101. In the illustrated embodiment, the actuatable objects 210, 212, 214, 216, 218 are in the form of virtual buttons that can be touched or depressed (via the touchscreen 103) for actuation. Any other suitable forms of actuation are contemplated, such as sliding buttons, sliding or swiping movements along the screen, etc.

In certain examples, a method of treating a patient can include using the generator 101 and selecting an operational mode displayed on the home screen 202. A method of controlling one or more of the RF probes 135a-d can involve receiving input from a user via one of the actuatable objects 210, 212, 214, 216, 218 on the home screen and transitioning the generator 101 to the selected operational mode in response to the user input via the control unit 109.

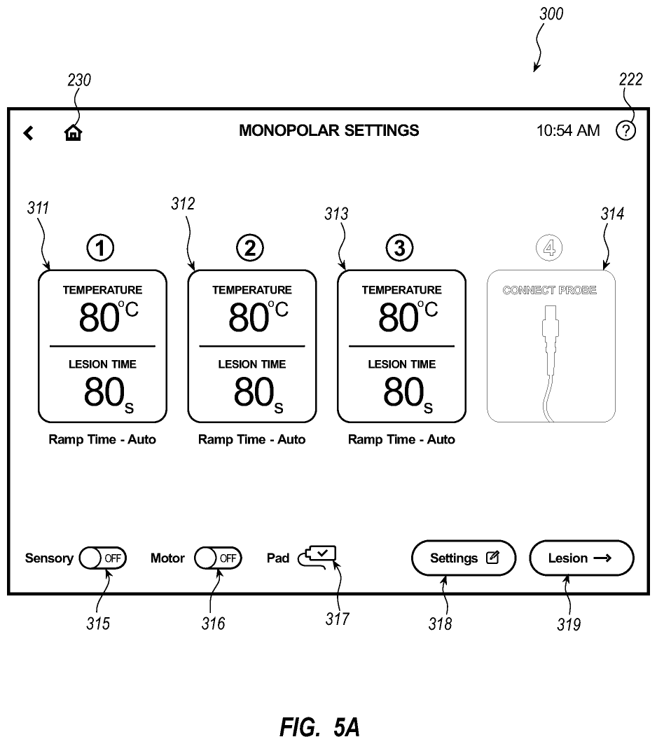

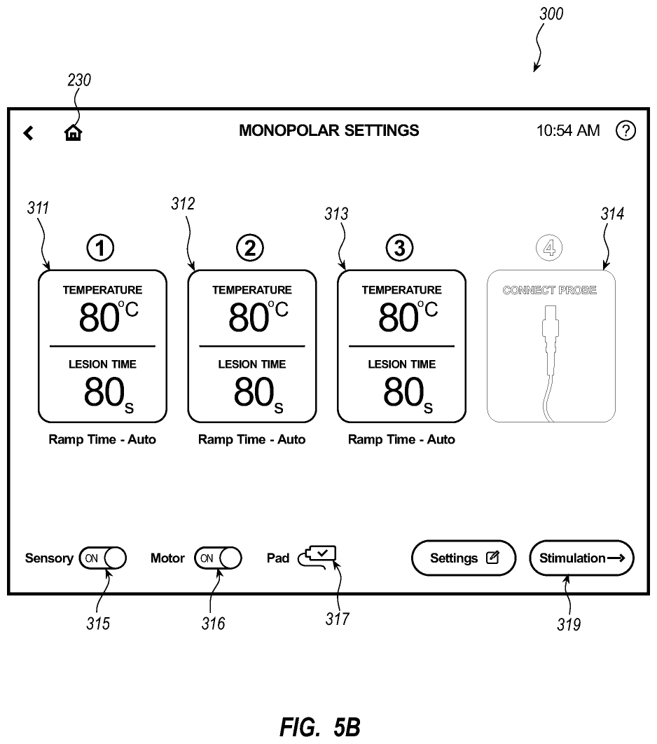

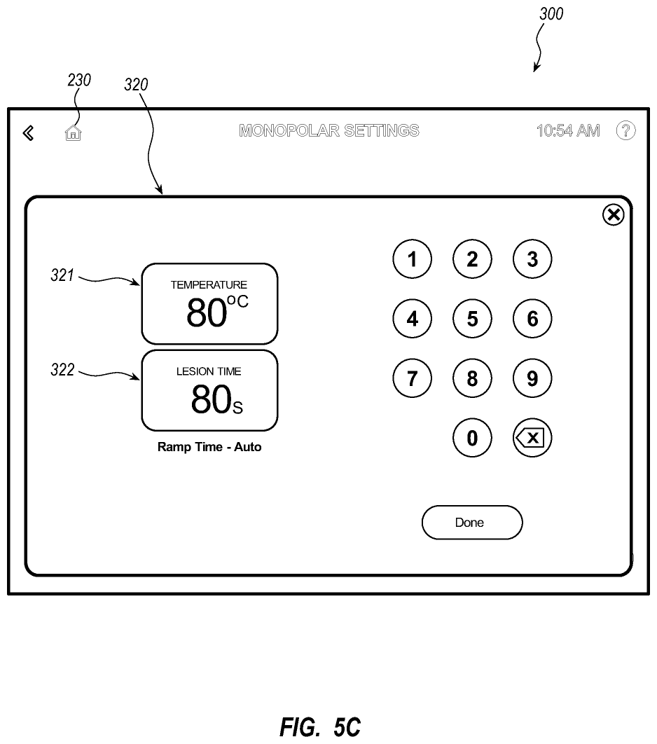

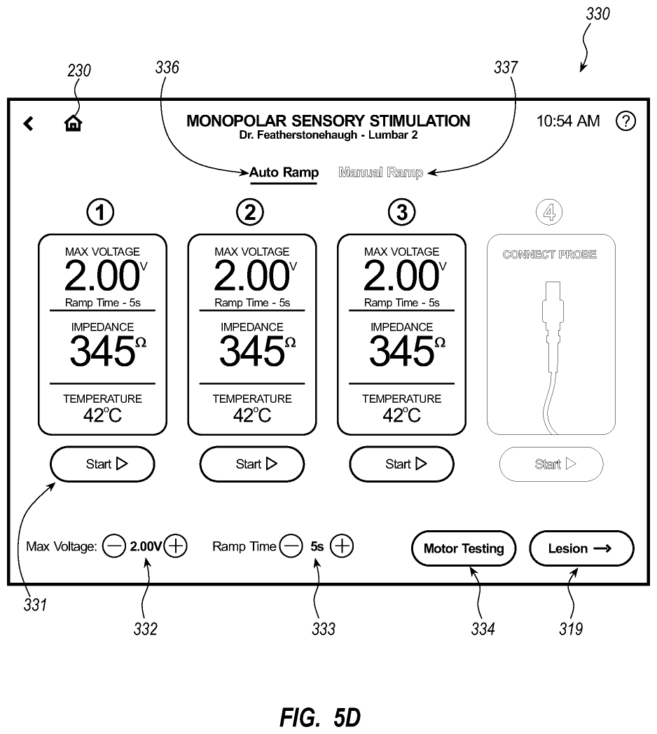

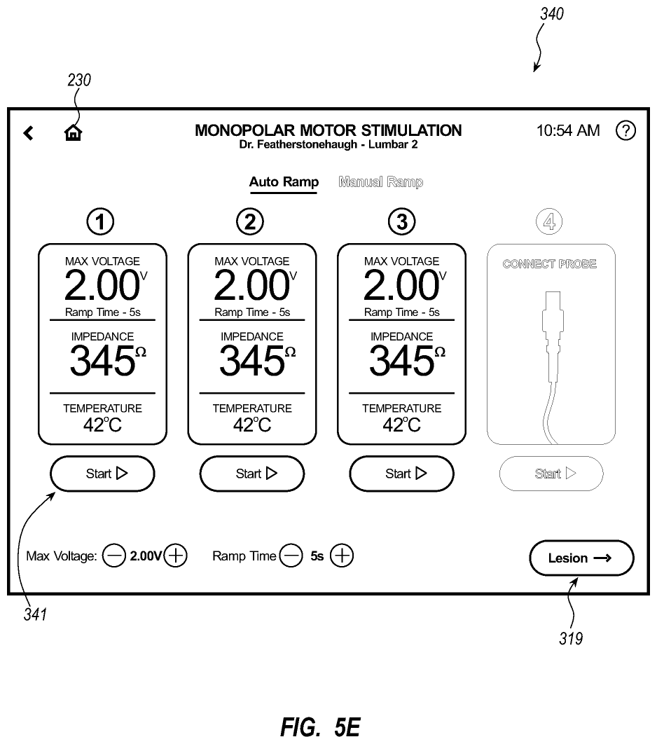

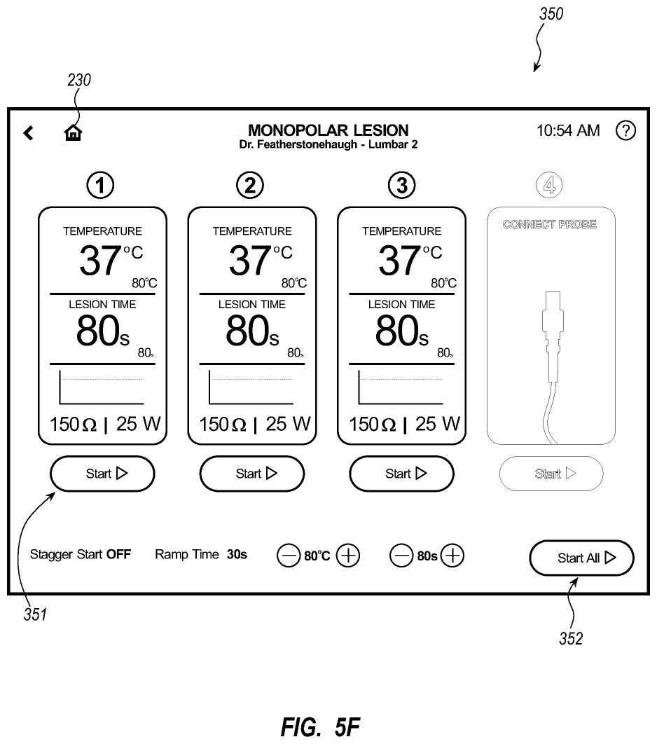

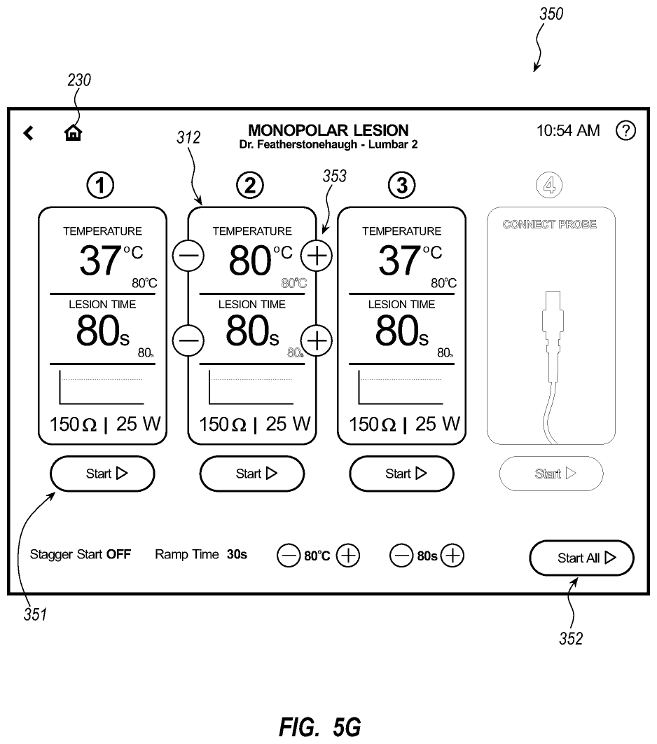

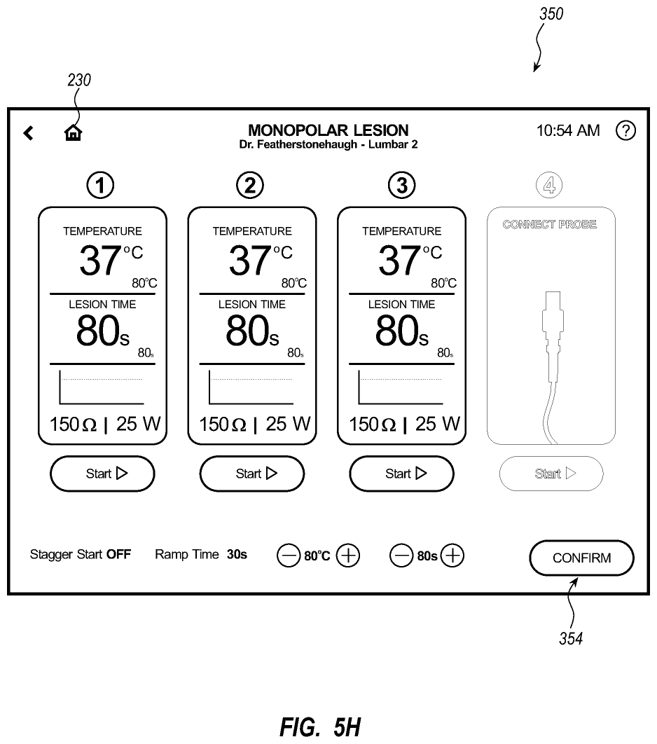

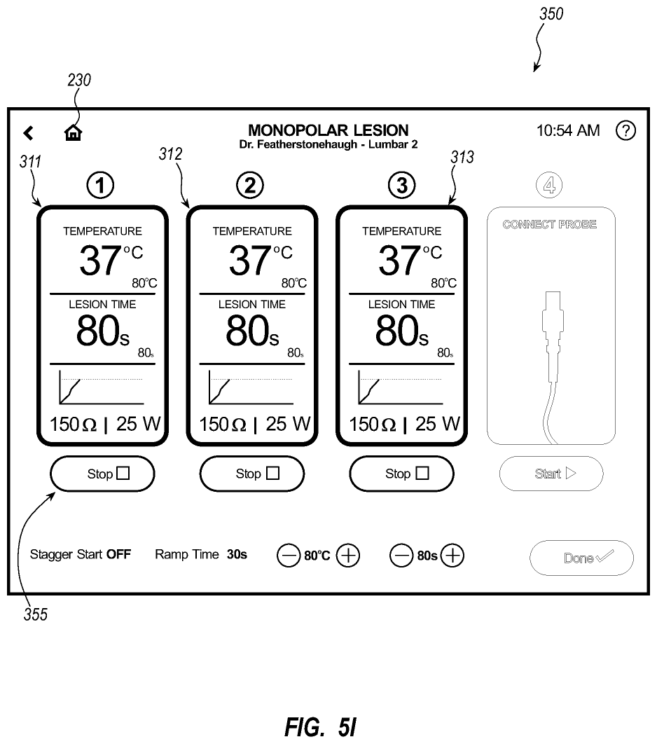

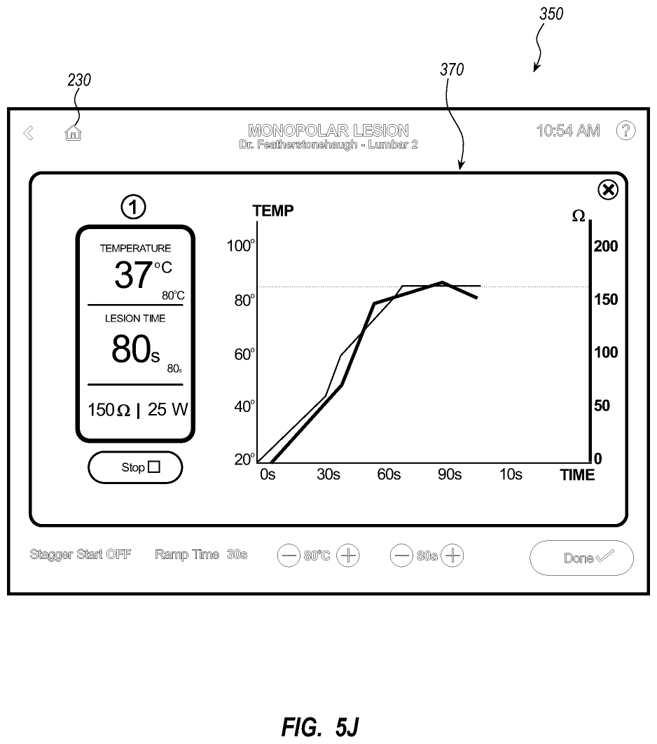

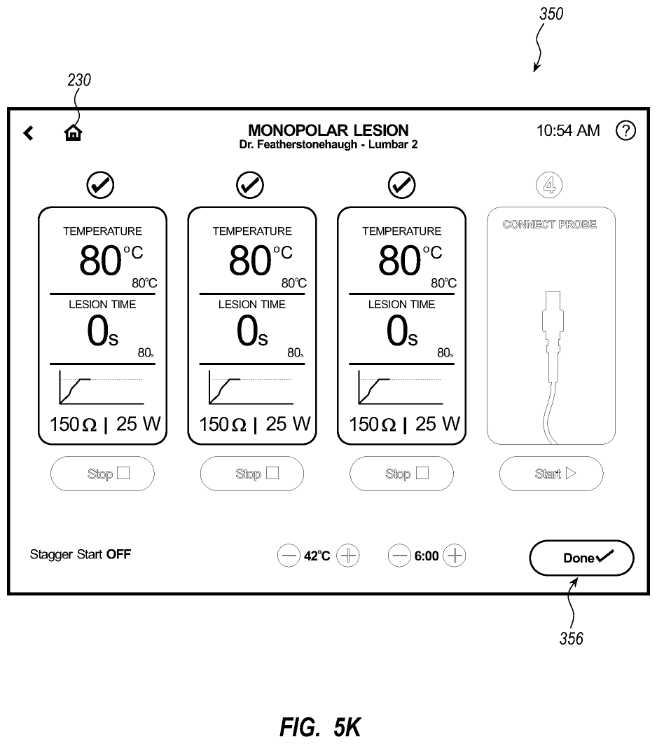

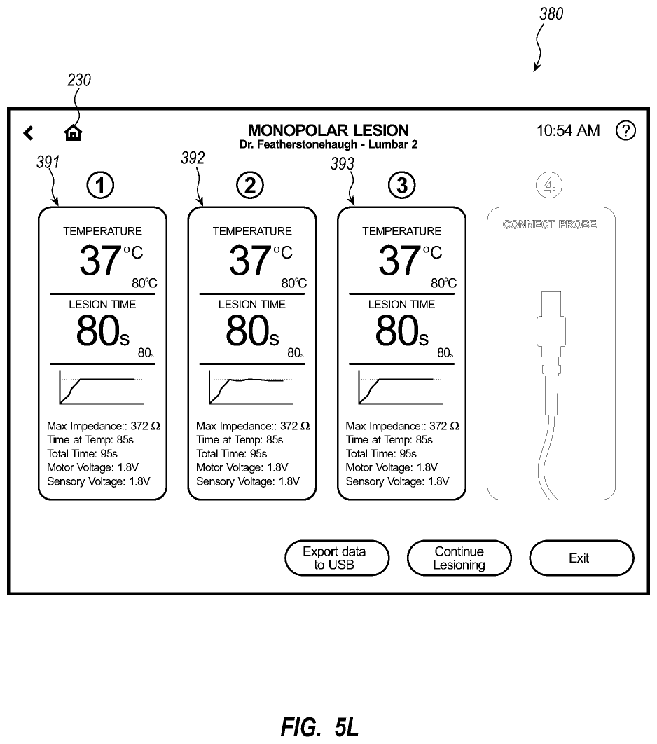

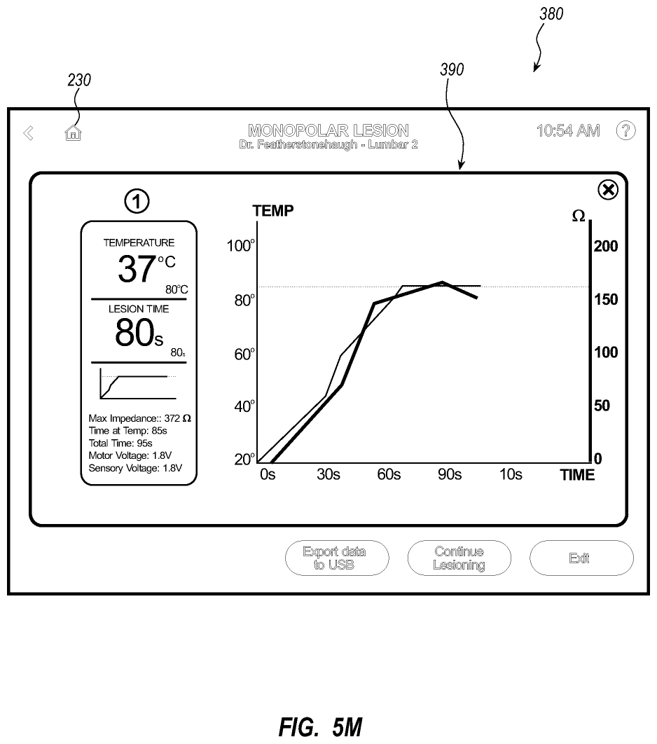

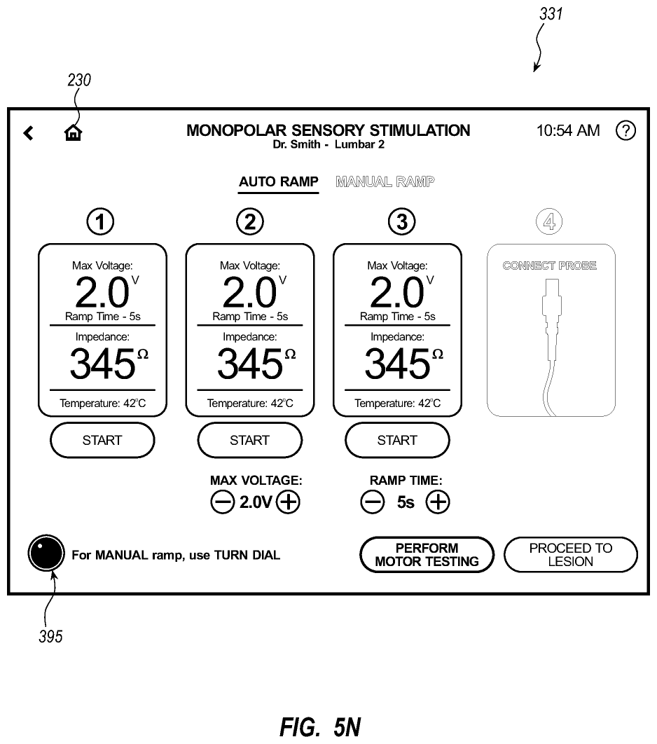

If a user selects actuatable monopolar procedure object 210, which is identified as option “1” on the home screen 202, the generator 101 is transitioned to a monopolar mode and a visual display 300, shown in FIG. 5A, is presented on the touchscreen 103. In particular, upon actuation of the object 210, the control unit 109 causes the touchscreen 103 to transition from the home screen 202 to the visual display 300, or stated otherwise, causes the home screen 202 to be replaced with the visual display 300.

In the illustrated embodiment, the visual display 300 is a monopolar settings screen. Various additional illustrative visual displays corresponding to the monopolar mode of the generator 101 are shown in and described below with respect to FIGS. 5A-5N.

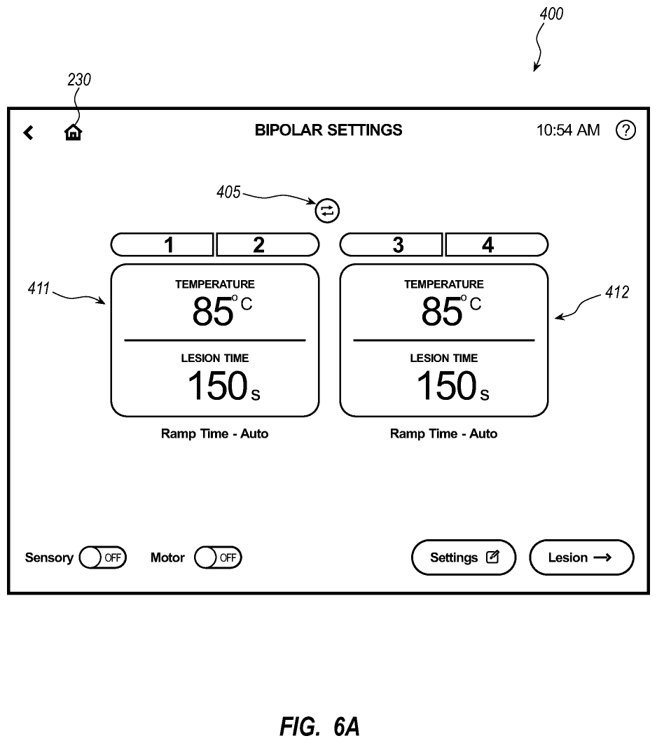

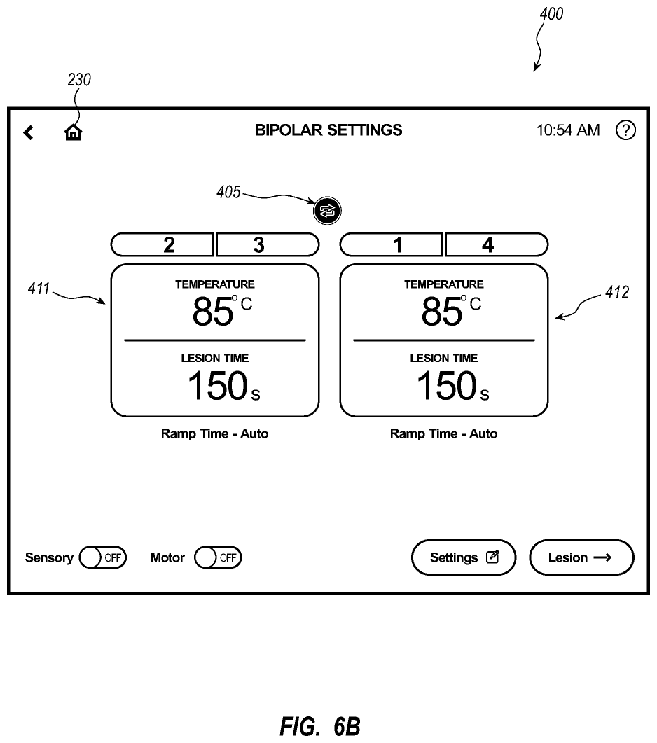

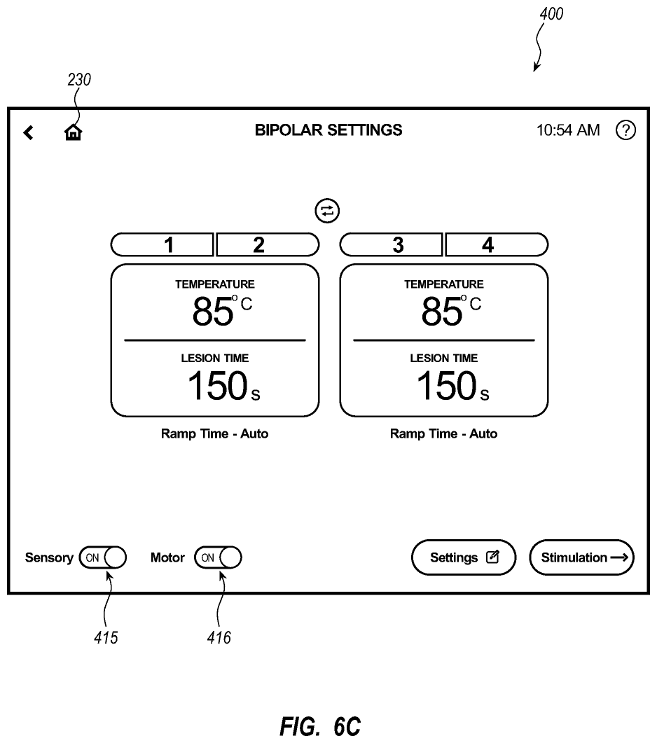

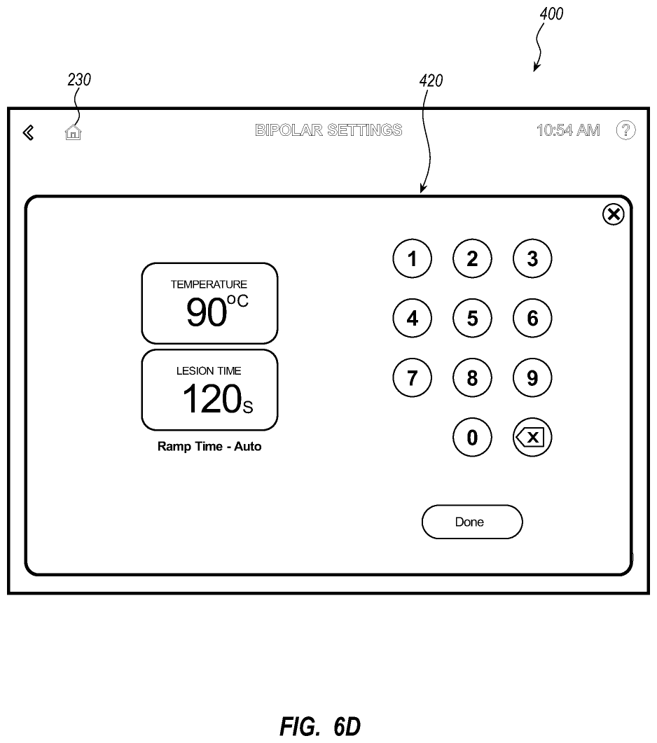

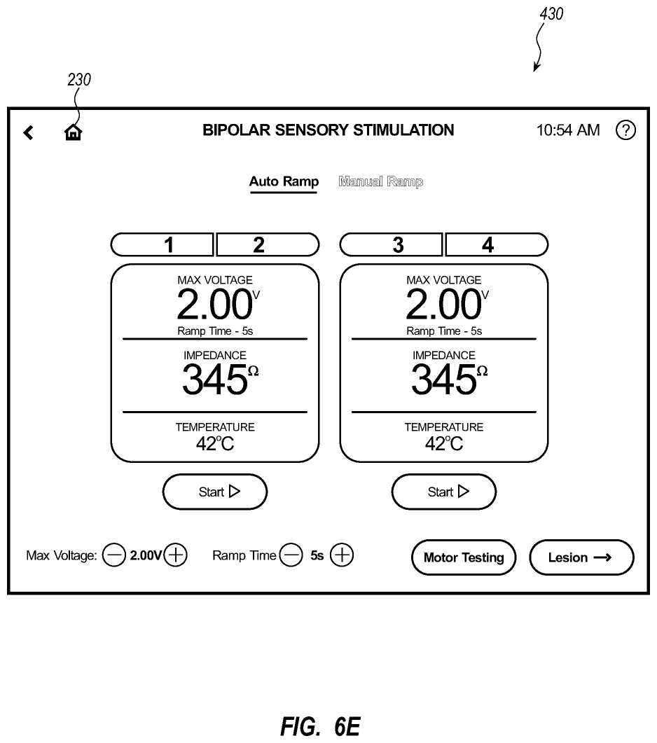

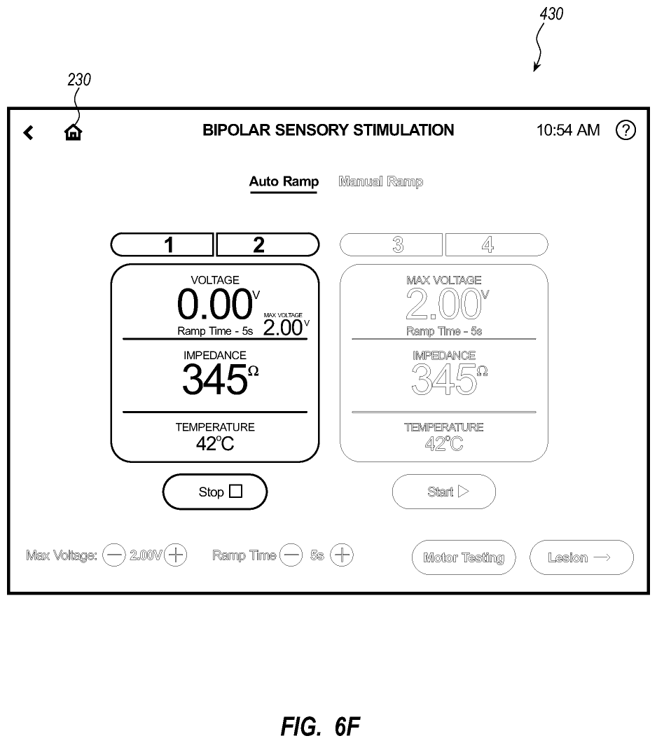

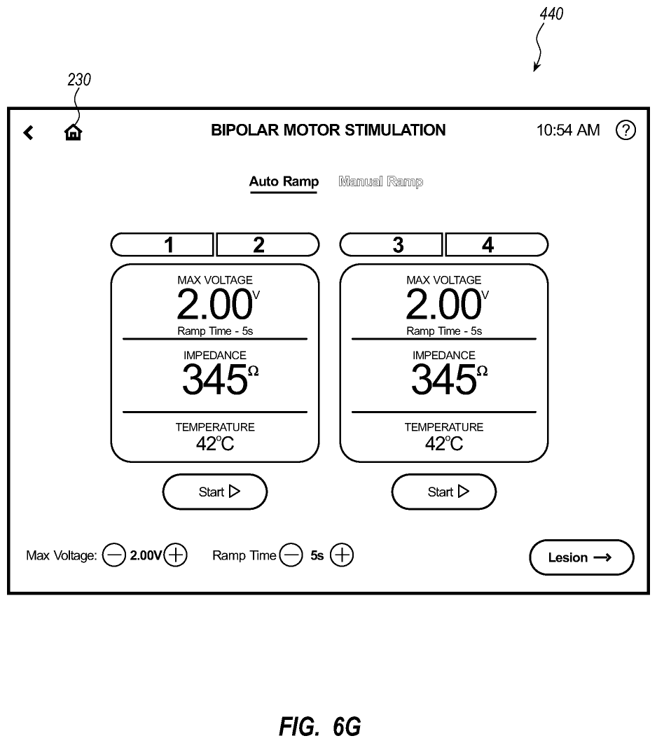

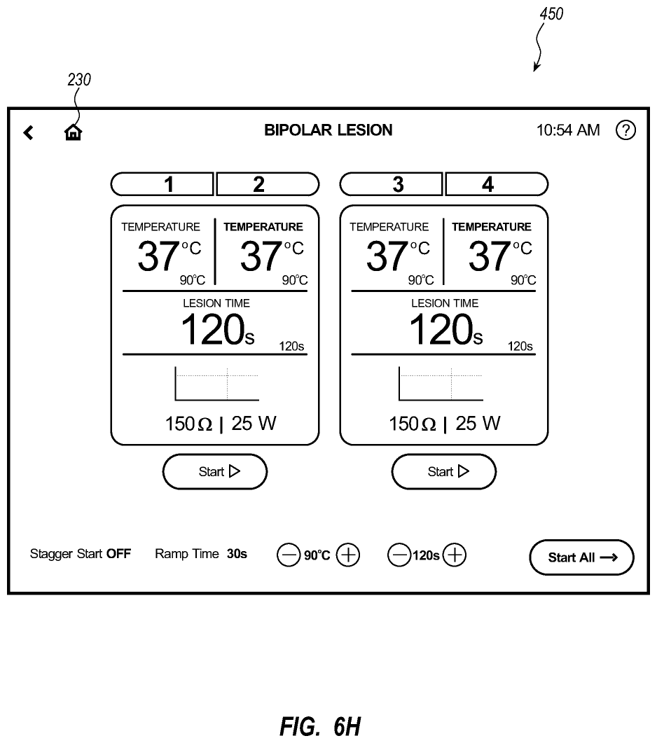

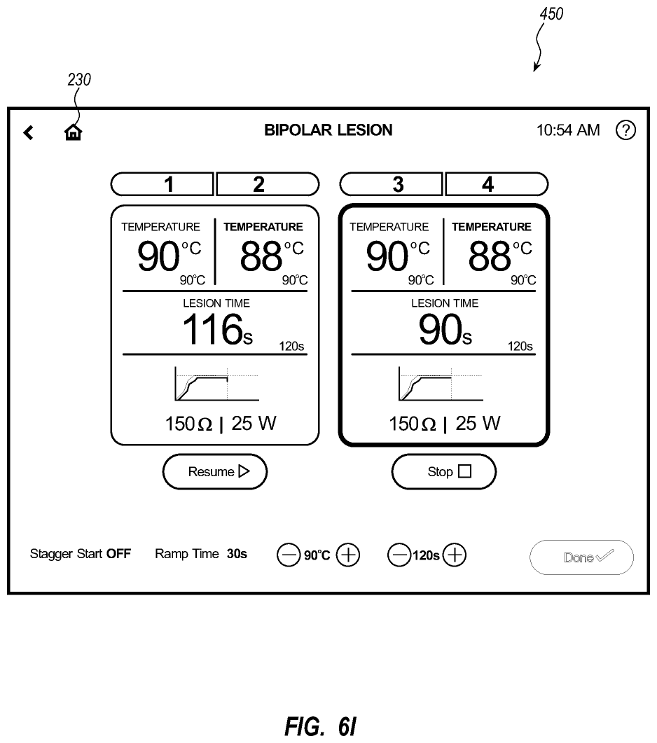

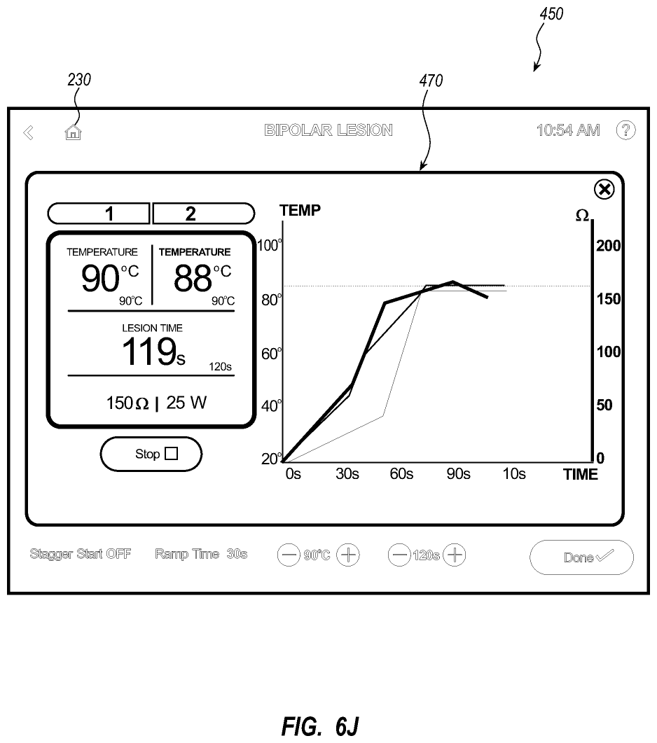

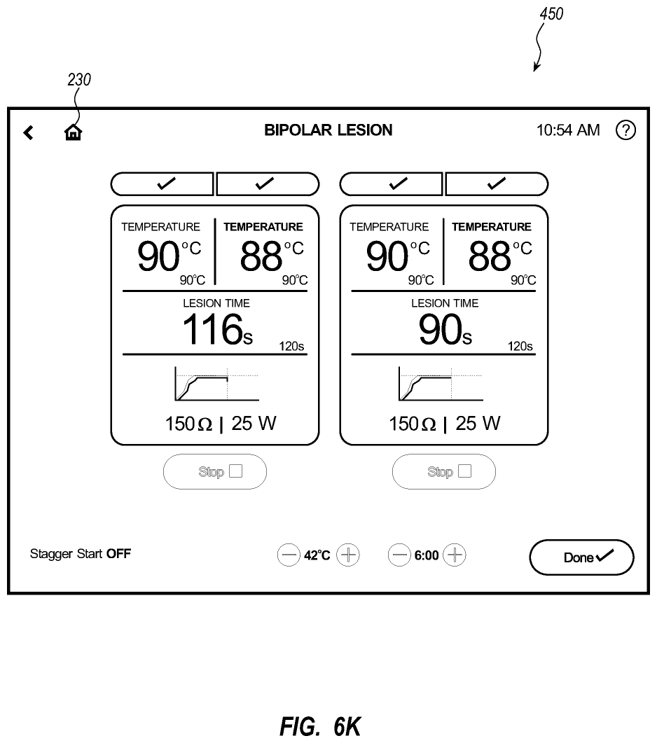

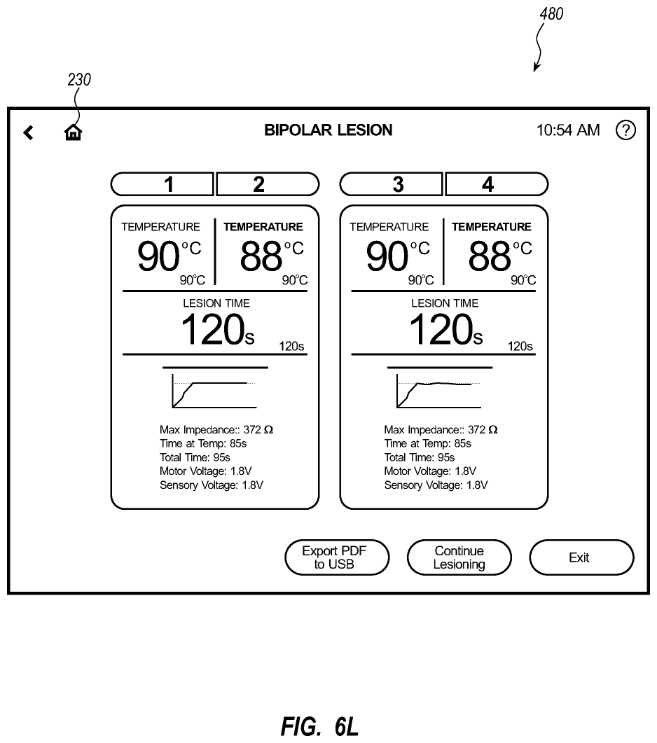

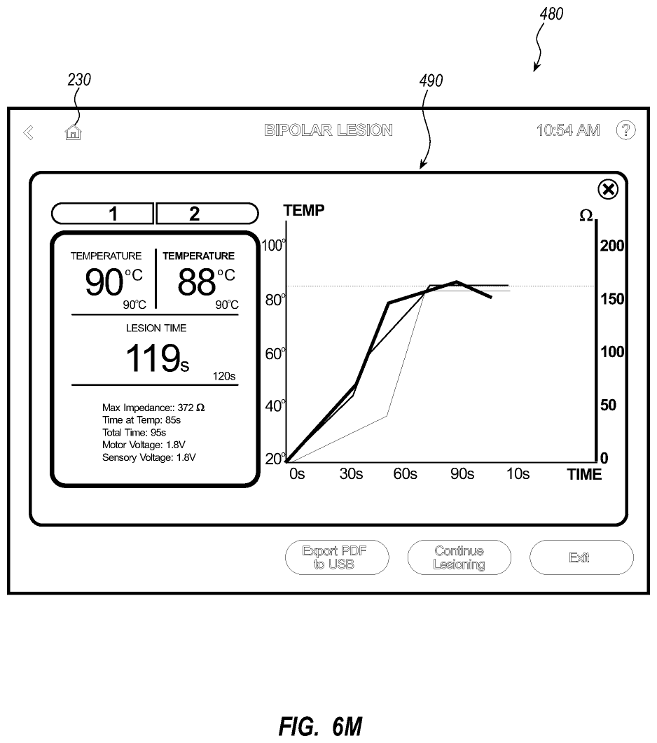

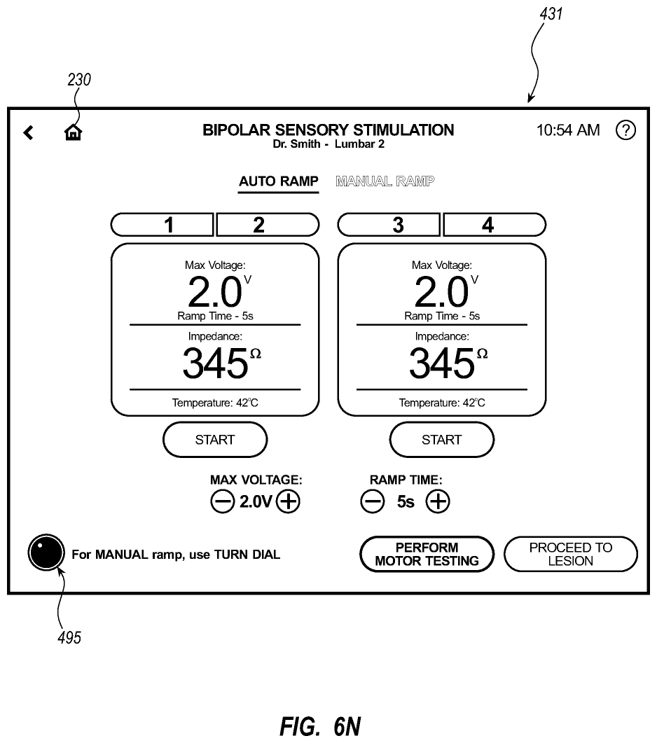

With continued reference to FIG. 4, if a user selects (e.g., presses) actuatable bipolar procedure object 212, which is identified as option “2” on the home screen 202, the generator 101 is transitioned to a bipolar mode and a visual display 400, shown in FIG. 6A, is presented on the touchscreen 103. Various illustrative visual displays corresponding to the bipolar mode of the generator 101 are shown in and described below with respect to FIGS. 6A-6N.

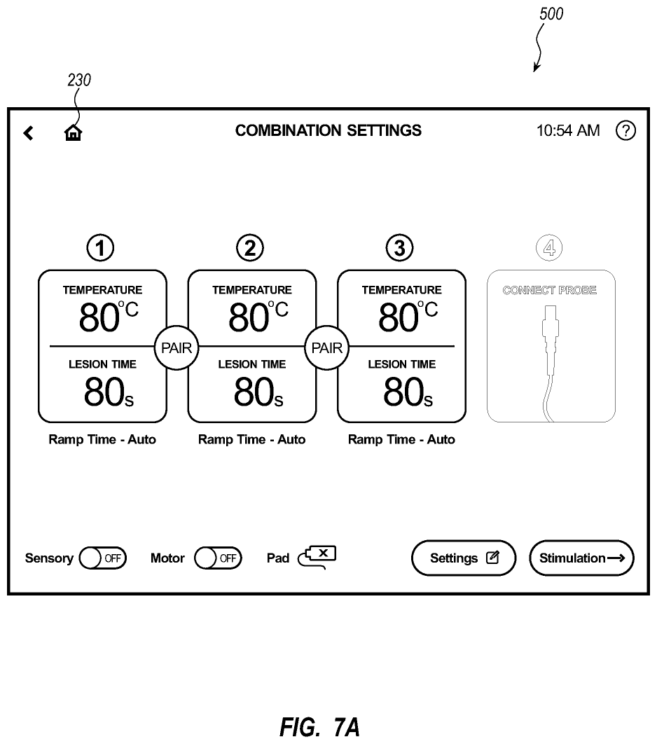

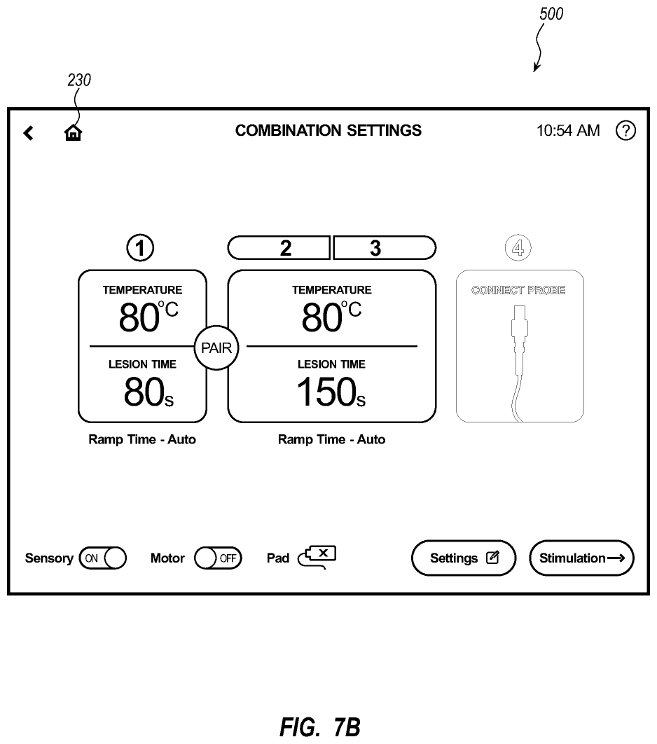

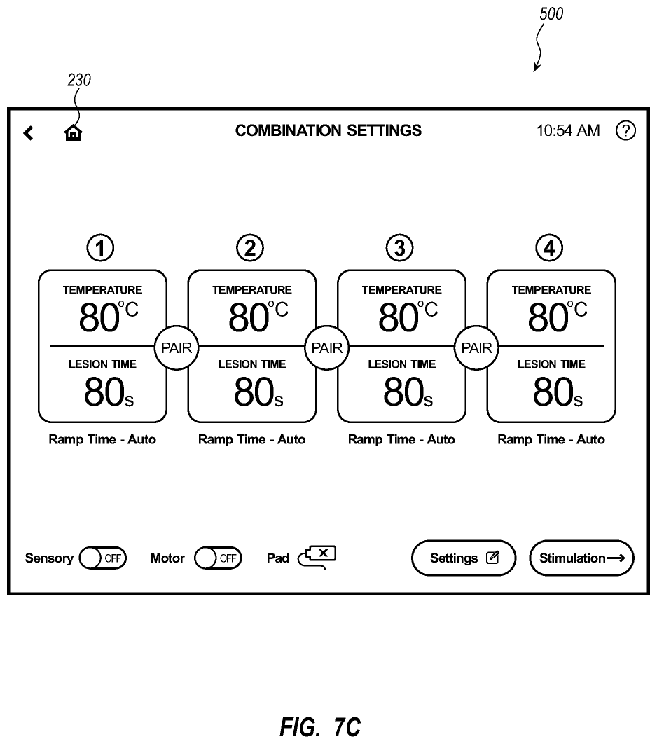

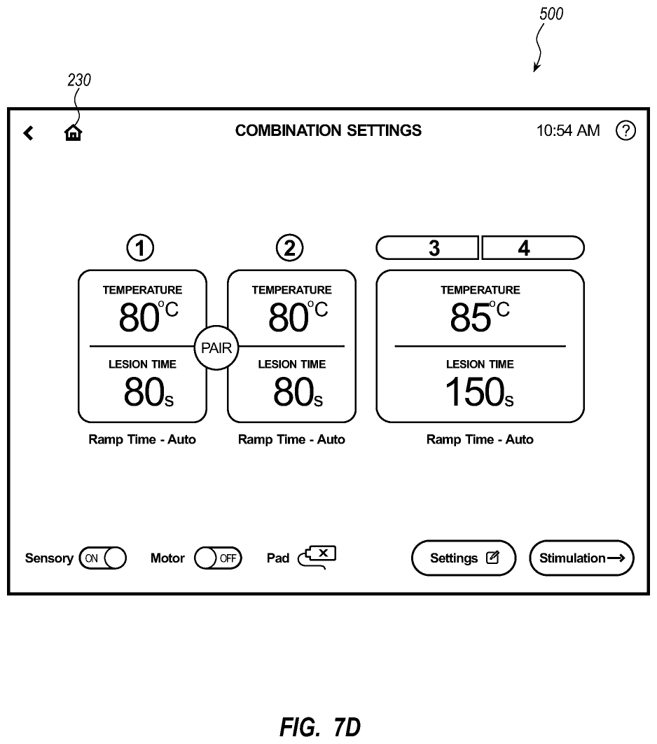

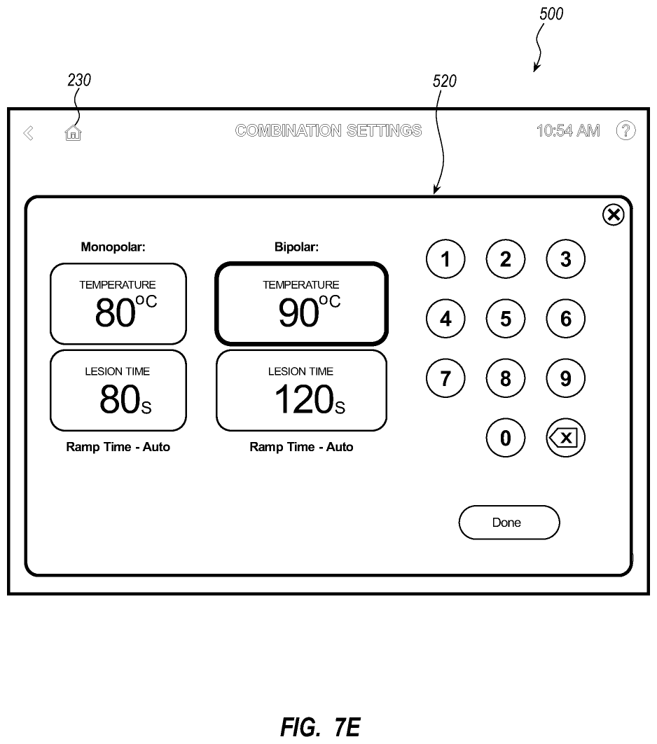

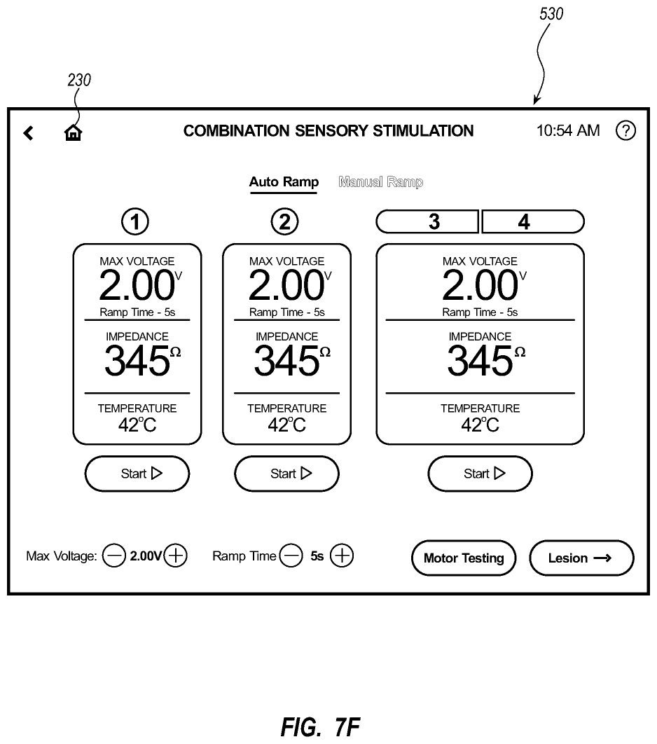

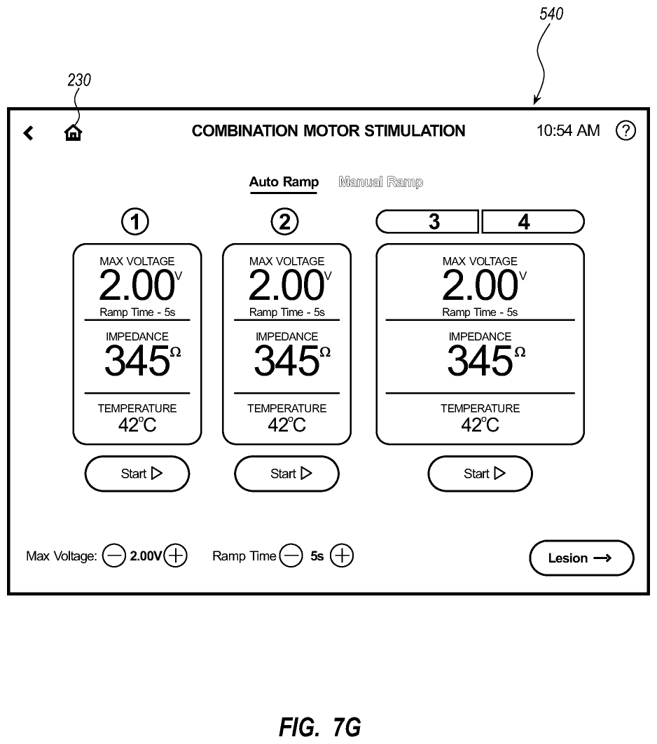

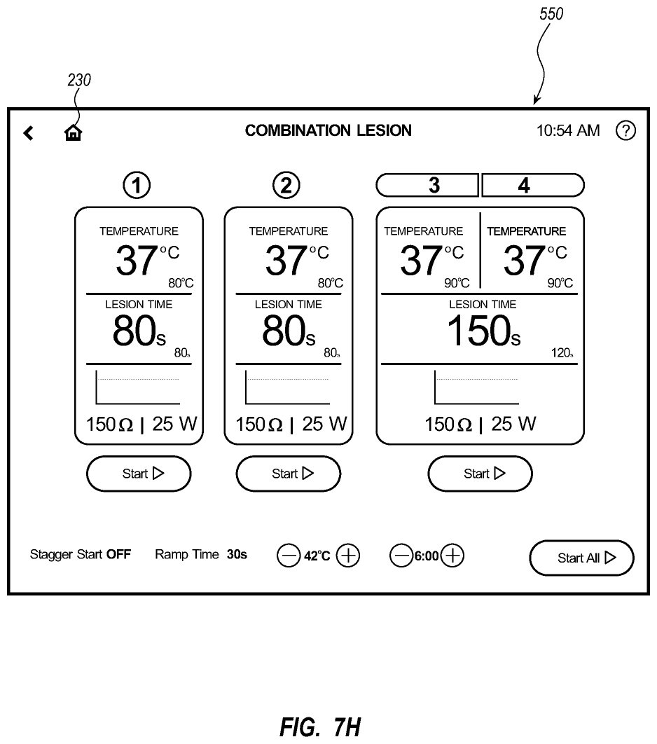

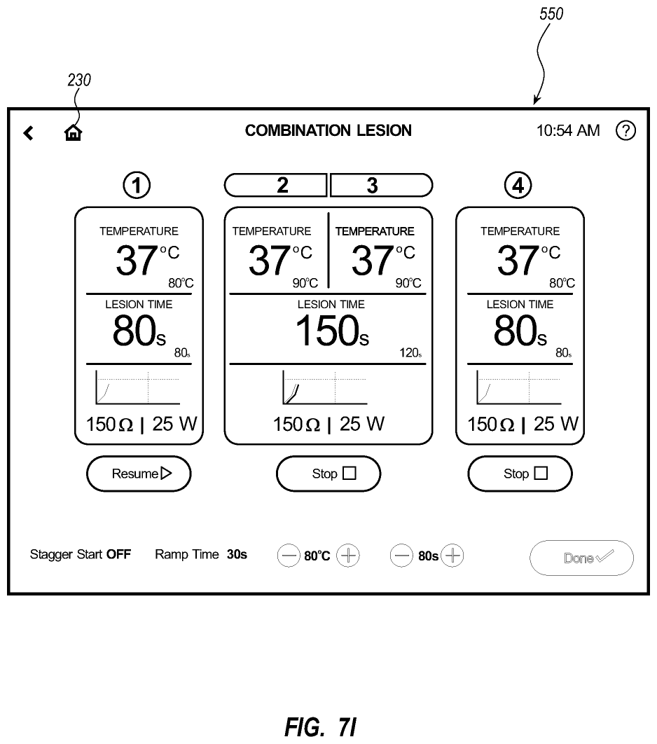

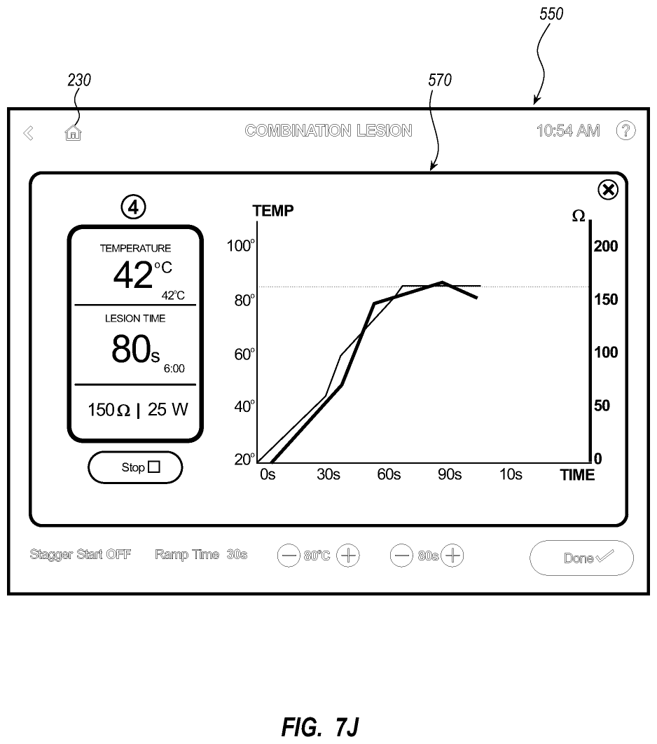

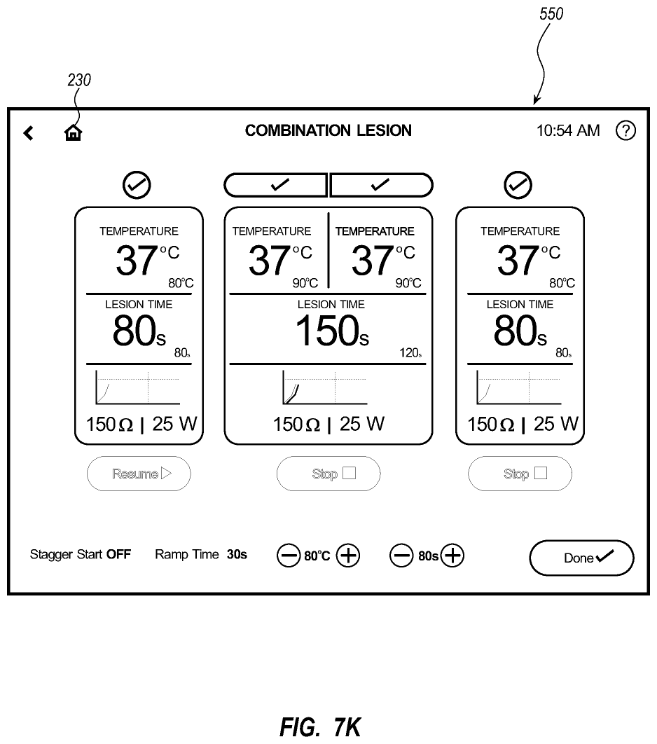

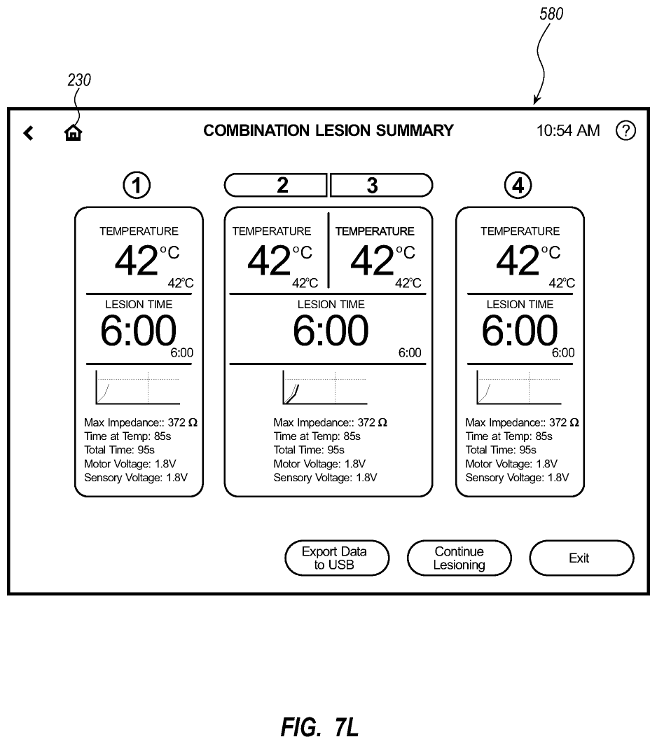

With continued reference to FIG. 4, if a user selects (e.g., presses) actuatable combination procedure object 214, which is identified as option “3” on the home screen 202, the generator 101 is transitioned to a combination mode and a visual display 500, shown in FIG. 7A, is presented on the touchscreen 103. Various illustrative visual displays corresponding to the combination mode of the generator 101 are shown in and described below with respect to FIGS. 7A-7L.

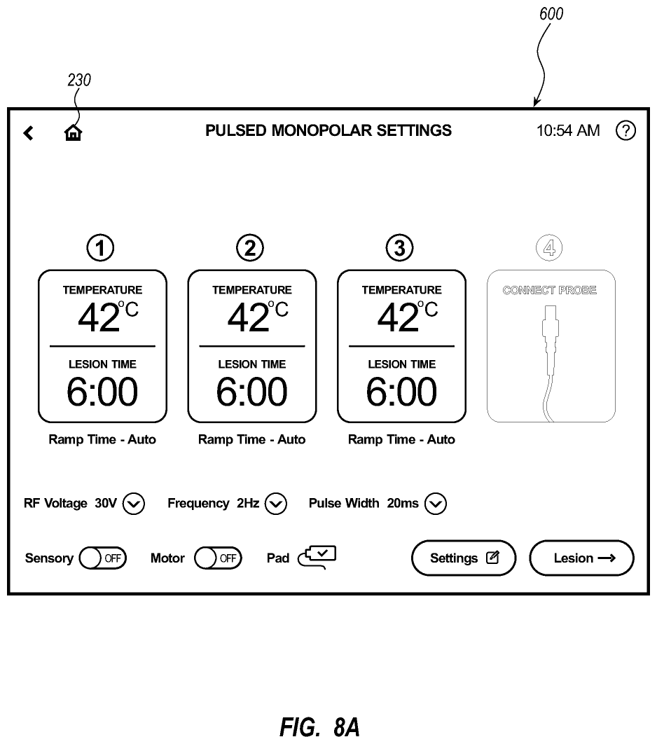

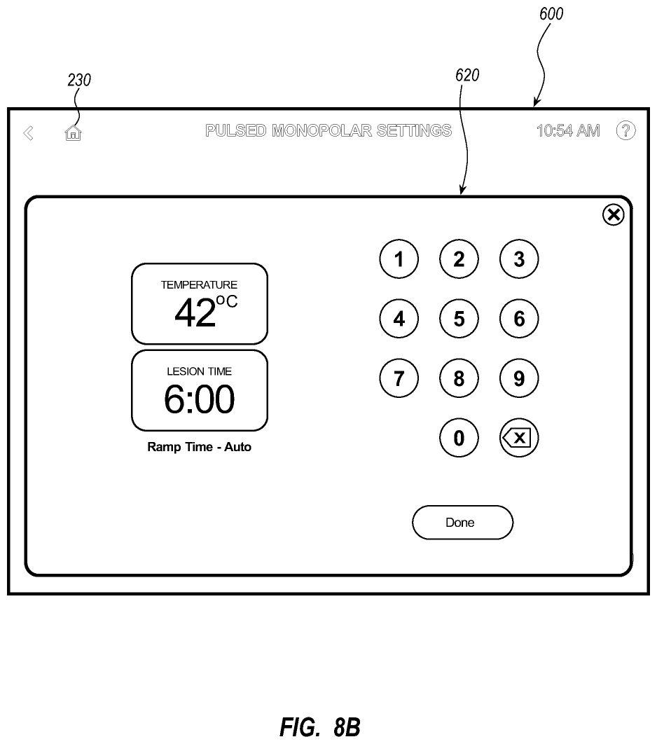

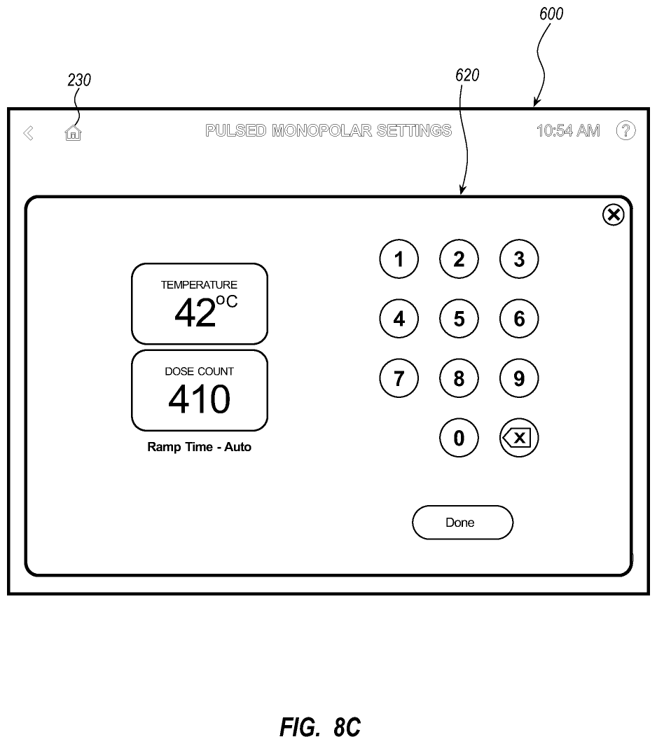

With continued reference to FIG. 4, if a user selects (e.g., presses) actuatable pulsed monopolar object 216, which is identified as option “4” on the home screen 202, the generator 101 is transitioned to a pulsed monopolar mode and a visual display 600, shown in FIG. 8A, is presented on the touchscreen 103. Various illustrative visual displays corresponding to the pulsed monopolar mode of the generator are shown in and described below with respect to FIGS. 8A-8F.

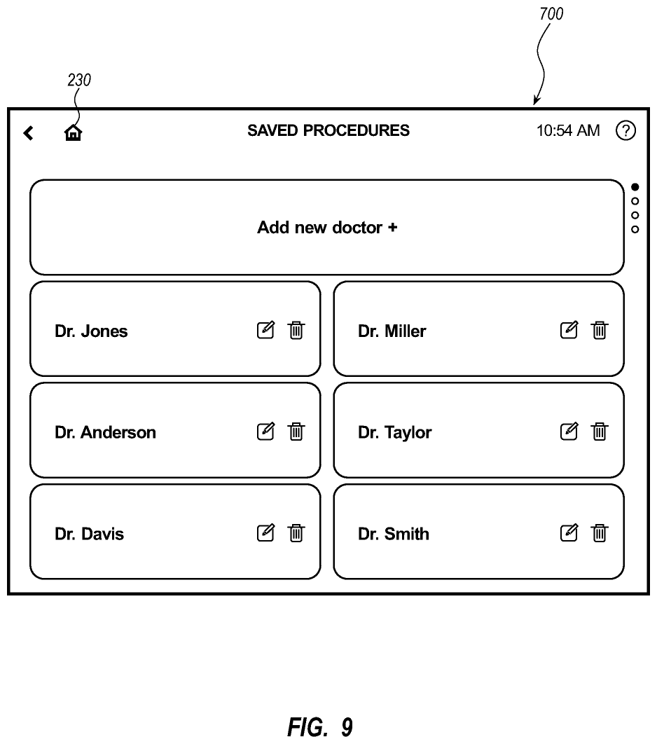

With continued reference to FIG. 4, if a user selects actuatable saved procedures object 218, which is identified as option “5” on the home screen 202, the generator 101 is transitioned to a saved procedure mode and a visual display 700, shown in FIG. 9, is presented on the touchscreen 103. The user is able to select from and/or store a variety of procedures in this mode, as discussed further below with respect to FIG. 9.

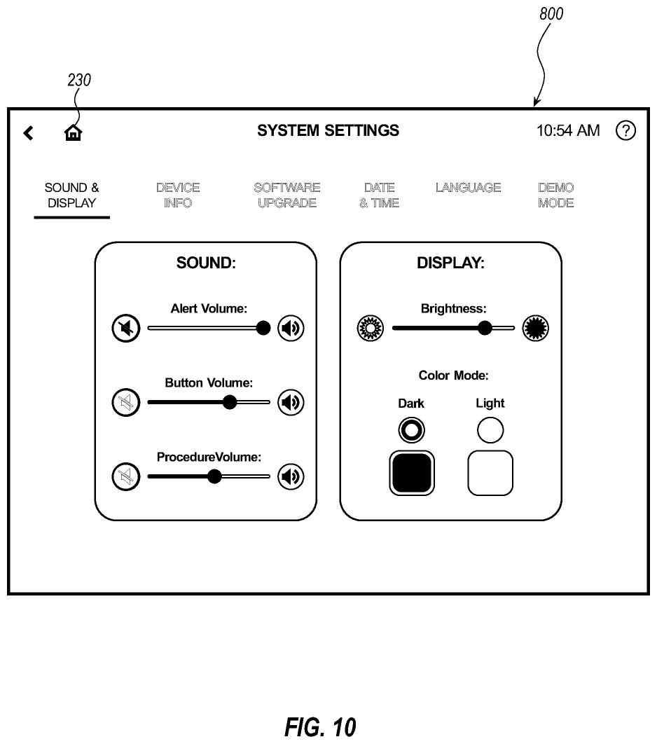

With continued reference to FIG. 4, if a user selects actuatable system settings object 220, which can include a gear icon or any other suitable settings icon, the generator 101 is transitioned to a settings mode and a visual display 800, shown in FIG. 10, is presented on the touchscreen 103. From this screen, the user is able to select a variety of preferred settings, obtain device information, upgrade software, and/or enter a demo mode, in certain embodiments, as discussed further below with respect to FIG. 10.

The home screen 202 can further include a contact object 221. A user can actuate the contact object 221 to activate a popup window or the like that provides relevant contact information. For example, contact information for the manufacturer of the generator 101 and/or contact information for a company representative may be present in the popup window.

With continued reference to FIG. 4, certain embodiments can include a help object 222 that can be selected to obtain help. In some instances, one or more popup windows may be shown when this virtual button is selected. The help provided may be contextual. In some instances, the virtual button 222 remains present on most or all visual displays (e.g., the visual displays 300, 400, 500, 600, 700, 800).

In various embodiments, the home screen 202 may be include features that enhance ease of use, for example, whether by new users who are relatively untrained in RF procedures or by experienced users. For example, in some instances, it may be helpful for the at least the objects 210, 212, 214 to be readily touched and/or readily viewable. In various embodiments, a combined area of the virtual buttons 210, 212, 214 collectively accounts for no less than ⅕, ¼, ⅓, or ½ of the displayable area of the touchscreen 202. In some embodiments, each of the first and second objects 210, 212 is bigger than the third object 214. In some embodiments, the first and second objects 210, 212 are the same size as each other.

In some embodiments, settings of the monopolar mode, the bipolar mode, and the combination mode are not directly editable on the home display 202. In some embodiments, at least one of the first object 210, the second object 212, the third object 214, or one or more additional objects (e.g., 216 or 218) must be actuated on the home display to cause at least one additional display to be shown on the touchscreen before edits to settings of any of the monopolar mode, the bipolar mode, or the combination mode can be made. In some embodiments, the home display 202 is devoid of visual indicia specifically associated or associable with any of the ports of the generator 101.

With reference to FIG. 5A, a homing object 230, which may also be referred to herein as a home button or home virtual button, may be present on most or all visual displays other than the visual display 200. For example, this virtual button 230 is present in the upper left corner of the screen in each of the visual displays 300, 400, 500, 600, 700, 800. Selecting this virtual button 230 will cause the visual display 200 or home screen 202 to be shown again. It may be said that the homing object 230 is displayed continuously once the generator 101 has entered into an operational mode from the home display 202. For example, in various embodiments, as shown in each of FIGS. 5A-5N, the homing object 230 may be present at all times on every screen displayed throughout a monopolar procedure; as shown in each of FIGS. 6A-6N, the homing object 230 may be present at all times on every screen displayed throughout a monopolar procedure; as shown in each of FIGS. 7A-7L, the homing object 230 may be present at all times on every screen displayed throughout a combination procedure; as shown in each of FIGS. 8A-8C, the homing object 230 may be present at all times on every screen displayed throughout a pulsed monopolar procedure; and as suggested in FIG. 9, the homing object 230 may be present at all times on every screen displayed throughout creating and/or accessing a saved procedure.

FIGS. 5A-5M depict a series of visual displays that correspond with the monopolar mode of an embodiment of the generator 101. Some or all of the visual displays can be shown on the touchscreen 103 and/or interacted with in one or more steps of illustrative methods of a monopolar RF procedure. In some instances, the visual displays may be shown and/or viewed in or substantially in the A-to-M sequence depicted. Other display orders are also possible.

As shown in FIG. 5A, the visual display 300 can correspond to a monopolar settings screen or display. Stated otherwise, the visual display 300 can be referred to as a monopolar settings display 300. Actuation of the monopolar procedure object 210 on the home screen 202 (see FIG. 4) can cause the screen sequence to proceed directly to the display 300.

The display 300 can include four distinct regions (labeled 1-4, respectively), each of which corresponds with a separate port 114a-d of the generator 101. For example, region 1 can correspond with port 114a, region 2 can correspond with port 114b, region 3 can correspond with port 114c, and region 4 can correspond with port 114d. In the illustrated arrangement, three RF probes (e.g., the probes 135a-c) are coupled with the ports 114a-c (see FIG. 1), whereas no RF probes are presently coupled with the fourth port 114d. Each probe region (1-4) can include an actuatable object 311, 312, 313, 314 that provides, e.g., temperature and lesion time information for a lesion procedure. Actuating the objects 311, 312, 313, 314, such as by pressing on virtual buttons, permits alteration of these settings.

In the illustrated embodiment, the visual display 300 includes toggle virtual buttons 315, 316 to allow selection of sensory and motor testing, respectively. In FIG. 5A, each of the sensory and motor buttons 315, 316 is unselected. Accordingly, a user may proceed directly to lesioning, as noted on the bottom righthand virtual button 319.

It is noted that the term “virtual button” or the like may be used with respect to certain actuatable objects in the present disclosure. It is understood that such references are not intended to be limiting. In other instances, virtual buttons could be replaced with any suitable actuatable object that can be actuated in any suitable manner, as discussed elsewhere herein.

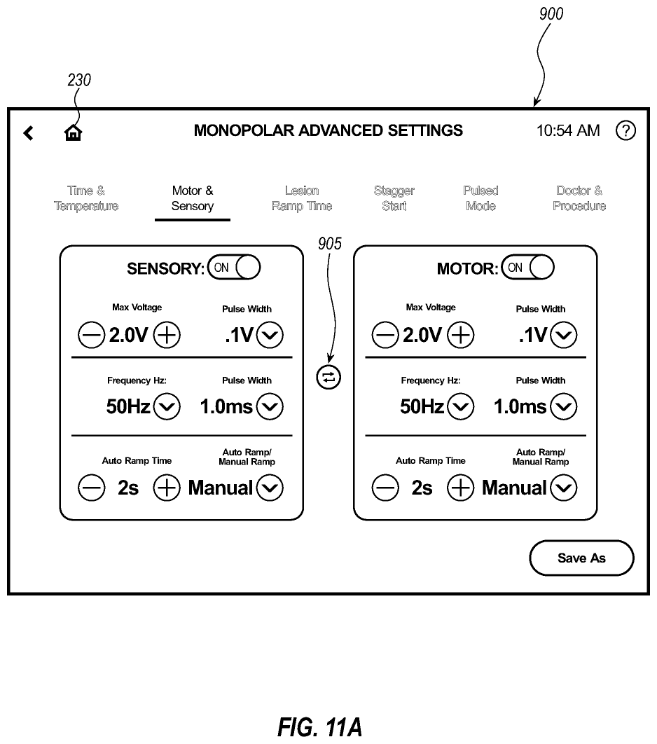

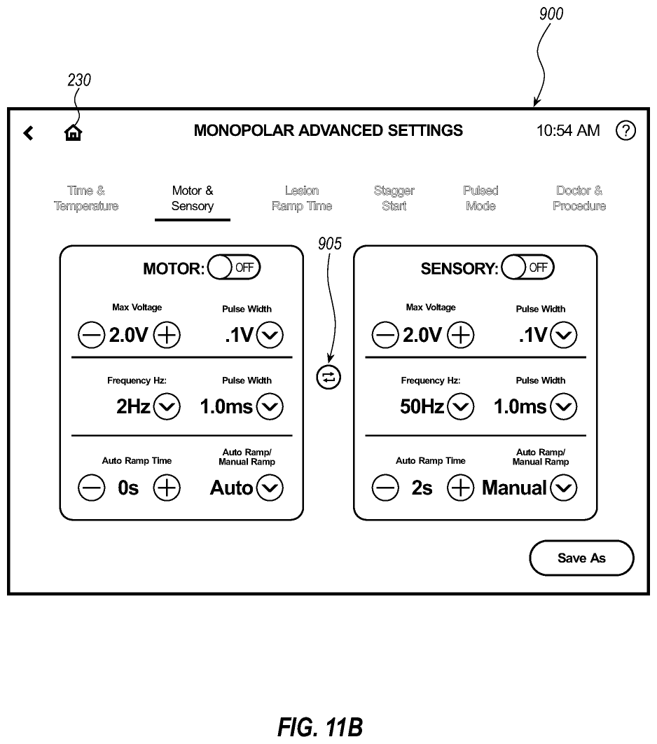

With continued reference to FIG. 5A, the display 300 can include a pad status object 317 that informs the user of a connection status of a grounding pad. In other or further instances, the display 300 can include a settings object 318 that can be actuated to proceed to advanced settings for the procedure at hand. In the present context, actuation of the settings object 318 can, for example, cause the control unit 109 to proceed to a monopolar advanced settings screen 900, such as that depicted in FIGS. 11A and 11B, which are discussed further below.

As shown in FIG. 5B, when the sensory and/or motor buttons 315, 316 are selected, the user is no longer provided with the option to proceed directly to lesioning at the actuatable object 319, but is instead allowed to proceed to the selected stimulation event or events. That is, the actuatable object 319 is now configured to proceed to the selected stimulation activity or activities, rather than proceeding to lesioning. In this and other manners discussed herein, the control unit 109, based on input from the user, dynamically adapts an RF procedure to the selections made by the user and helps step the user through the procedure.

FIG. 5C depicts a popup display 320 that appears over the display 300 when any of the objects 311, 312, 313 is selected. The popup display 320 permits the user to readily adjust the temperature and lesion time for the monopolar procedure. For example, the user can press on the virtual button 321 to adjust the temperature setting using the virtual number pad and/or the user can press on the virtual button 322 to adjust the lesion time setting using the virtual number pad. When all desired selections and adjustments have been made, the user can close the popup display 320 via the “x” button in the upper right corner of the display 320, which then returns the user to the unobstructed display 300 as shown in FIG. 5B.

FIG. 5D depicts a visual display 330 that is shown upon pressing the “stimulation” button 319 as provided in FIG. 5B. Various options are provided to the user by way of further virtual buttons. As noted in the upper title bar, “MONOPOLAR SENSORY STIMULATION” is the first available stimulation event. The user can select the appropriate “Start” buttons 331 to start the sensory stimulation for each individual probe. Maximum voltage and ramp time may be altered via the appropriate virtual buttons in the regions 332, 333 at the bottom of the screen. From this screen, the user has the option to either move on to further stimulation—that is, to go to “Motor Testing” by pressing the virtual button 334—or to skip the motor testing and proceed directly to lesioning by pressing the “Lesion” button 319.

As shown above the probe regions of the display, the default setting is an automatic ramping of voltage (as shown by the “Auto Ramp” indicator 336) for the sensory stimulation test. Alternatively, a user can automatically transition the ramping to manual control by turning the dial 107 (FIG. 1). Stated otherwise, mere turning of the dial 107 provides sufficient information or instruction to the control unit 109 (FIG. 3) for the control unit 109 to transition voltage ramping from an automated mode to a manual mode. In the manual mode, the “Manual Ramp” indicator 337 will be highlighted and underlined on the visual display, rather than the “Auto Ramp” title (which is underlined and highlighted in FIG. 5D due to the generator 101 being in the automatic ramping mode). The user can control the speed at which voltage is increased by rotating the dial 107 at a desired rate.

With reference to FIG. 5N, in alternative versions of various screens described herein, such as an alternative of the screen 330 of FIG. 5D, one or more screens 5N can include a visual prompt 395 to instruct a user that to effect manual ramping, the user can use the turn dial 107.

Returning to FIG. 5D, a user can press the “Motor Testing” object 334 to proceed to motor testing. This activity can cause the control unit 109 to make the touchscreen 103 show the display 340 depicted in FIG. 5E.

The display 340 of FIG. 5E provides various options to the user by way of further virtual buttons. As noted in the upper title bar, “MONOPOLAR MOTOR STIMULATION” is the stimulation event at hand. The user can select the appropriate “Start” buttons 341 to start the motor stimulation for each individual probe. Max voltage and ramp time may be altered via the appropriate buttons at the bottom of the screen.

As with sensory stimulation, and as shown above the probe regions of the display, the default setting is an automatic ramping of voltage for the motor stimulation test. Alternatively, a user can automatically transition the ramping to manual control by turning the dial 107 (FIG. 1). Stated otherwise, mere turning of the dial 107 provides information or instruction to the control unit 109 (FIG. 3) for the control unit 109 to transition voltage ramping from an automated mode to a manual mode. In the manual mode, the “Manual Ramp” title will be highlighted and underlined on the visual display, rather than the “Auto Ramp” title (which is underlined and highlighted in FIG. 5E due to the generator 101 being in the automatic ramping mode). From this screen 340, the user can move on to a lesioning mode when desired by pressing the virtual button 319, which in this instance is labeled “Lesion”.

FIG. 5F depicts a visual display that is shown upon pressing the “Lesion” button 319 of, e.g., FIG. 5E. Various options are provided to the user by way of further virtual buttons. Monopolar lesioning via each RF probe 135a-c may be started individually via the “Start” buttons 351, or all probes 135a-c may be started simultaneously via the “Start All” button 352.

FIG. 5G depicts the visual display 350 of FIG. 5F after the large virtual button 312 associated with the second RF probe is pressed. Virtual “+” and “−” buttons appear alongside each of the temperature and lesion time fields. The user can fine-tune these setting prior to proceeding with lesioning.

Once all desired settings are in order, a user can press the individual “Start” buttons or the “Start All” button in the bottom righthand corner of the display, as desired.

FIG. 5H depicts the visual display 350 as shown upon pressing the “Start All” button 352 of FIG. 5G. As a safety measure, the “Start All” button 352 is updated to a “Confirm” button 354 to ensure that the user truly intends to start lesioning with all devices.

FIG. 5I depicts a visual display that is shown upon pressing the “Confirm” button 354 of FIG. 5H. The simultaneous lesioning via all three probes that are connected to the first three ports of the generator 101 is under way, with real-time graphical displays of temperature measurements obtained at the distal end of each probe shown beneath the “Lesion Time” region of each object 311, 312, 313. The user is provided the option to stop lesioning at any or all probes via the virtual “Stop” buttons 355. In some embodiments, the generator 101 includes a dedicated physical “Stop All” button that may also be used to stop all lesioning. In some embodiments, a halo effect or other highlighting or indicium is applied to the objects 311, 312, 313 associated with each active probe to visually indicate to a user that lesioning is underway.

FIG. 5J depicts a popup display 370 that can cover a portion of the display 350. The display 370 provides a more detailed view of the ongoing monopolar procedure associated with the first port of the generator 101. The popup display 370 can be summoned by pressing on the virtual button 311 in FIG. 5I as the procedure is ongoing. The display 370 can be dismissed by pressing on the “X” button at the top right hand side of the display 370. Similar detailed displays can be summoned for any of the active buttons 312, 313 (see FIG. 5I) during the procedure.

With continued reference to FIG. 5J, it is noted that the popup display 370 does not cover the homing object 230. This is also true of other popup displays (see FIG. 5C). In some embodiments, the homing object 230 is shown continuously throughout a selected procedure, and a user can return to the home screen 202 at any time during the procedure.

As shown in FIG. 5K, upon completion of the lesioning, a “Done” button 356 appears. The user can press this button to proceed to display 380 of FIG. 5L, which provides a summary of the procedure. At this screen, the user is provided with the options to export data to a USB, continue lesioning, or exit via buttons provided at the bottom of the screen. In some instances, actuating the “Exit” button can return the user to the home screen 202 of FIG. 4.

Further, as previously noted, if at any point a user desires to operate the generator 101 in a different mode, the user can press the home button at the top left corner to return to the home screen of FIG. 4.

FIG. 5M depicts a popup display 390 that can cover a portion of the display 380. The display 390 provides a more detailed view of the completed monopolar procedure associated with the first port of the generator 101. The popup display 390 can be summoned by pressing on the virtual button 391 of the display 380 shown in FIG. 5L. The display 390 can be dismissed by pressing on the “X” button at the top right hand side of the display 390. Similar detailed displays can be summoned for any of the buttons 392, 393 (see FIG. 5L).

As previously discussed, FIG. 5N depicts an alternative embodiment of a monopolar sensory stimulation screen 331.

FIGS. 6A-6M depict a series of visual displays that correspond with the bipolar mode of an embodiment of the generator 101. Some or all of the visual displays can be displayed and/or interacted with in one or more steps of illustrative methods of an RF procedure. In some instances, the visual displays may be shown and/or viewed in the A-to-M sequence depicted. Moreover, many of the items included in these visual displays, such as, e.g., titles and actuatable objects, closely resemble features previously discussed with respect to the visual displays for monopolar procedures. Accordingly, discussions of such features may not be repeated or fully addressed hereafter. It will be readily understood how the previous discussions apply to such features depicted in FIGS. 6A-6M. In some instances, like features between FIGS. 5A-5M and FIGS. 6A-6M are identified with like reference numerals, with the leading digit having been incremented. This pattern of disclosure is also used throughout the remainder of this description, where applicable.

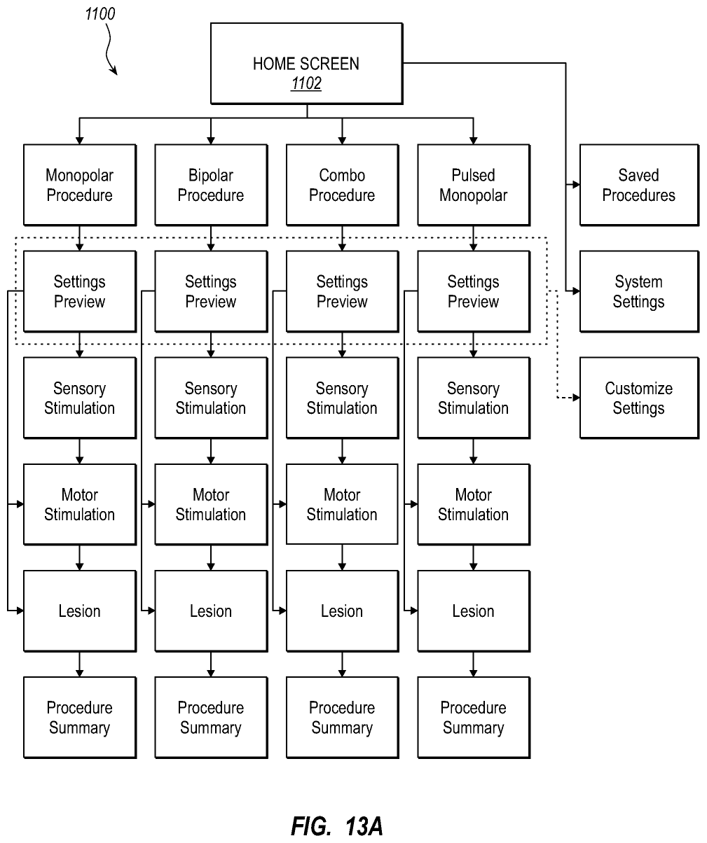

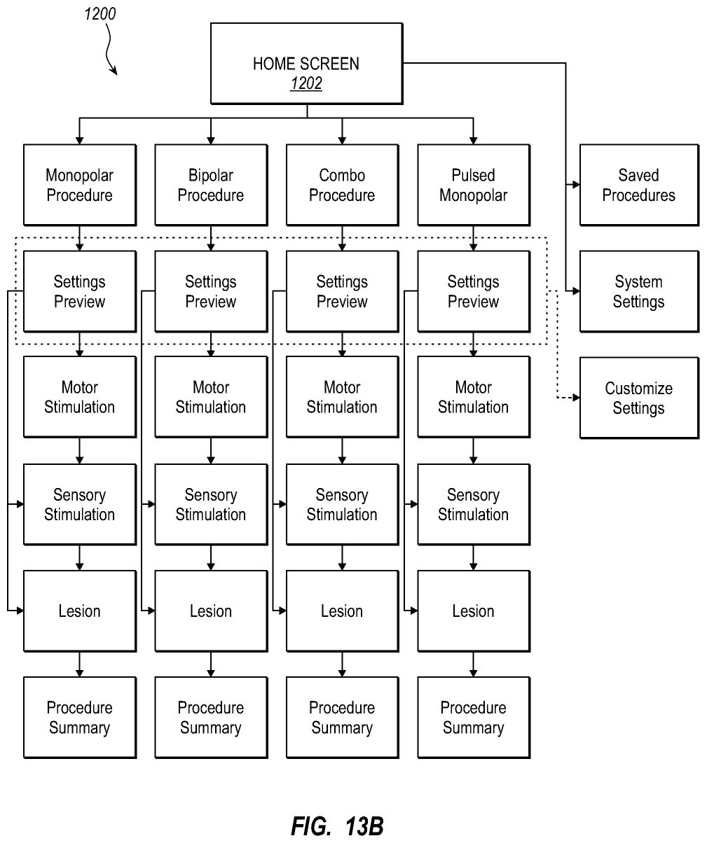

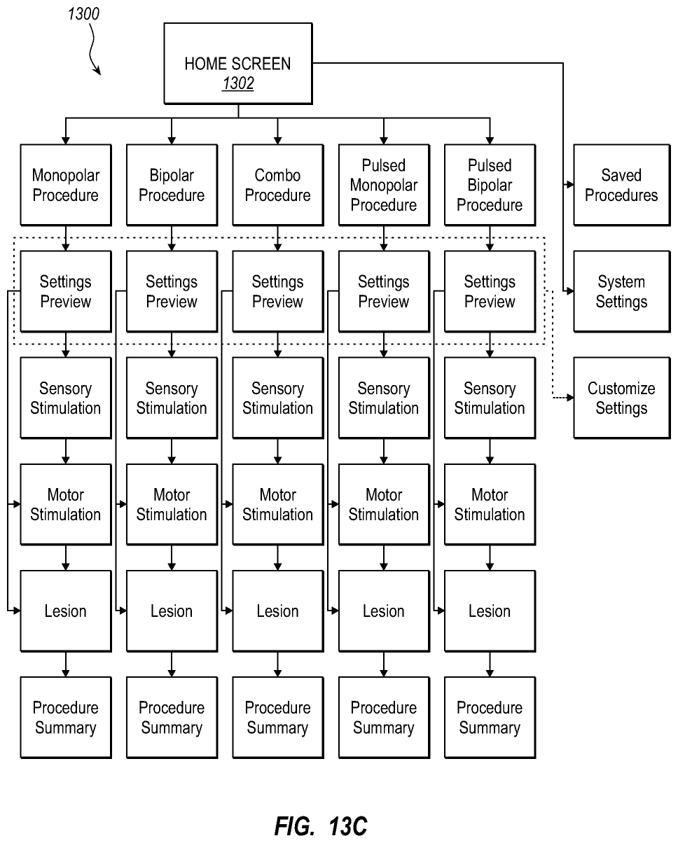

Further, as discussed further below with respect to FIGS. 13A-130, in many instances different types of procedures that can be performed via the generator 101 may follow substantially parallel paths, or stated otherwise, can have similar steps or stages. For example, the home screen 202 can provide for the selection of any of a variety of different procedures, yet upon selection of any one procedure, the generator 101 can lead a user along a similar sequence of steps, stages, or screens for some or all of the different procedures.

As shown in FIG. 6A, the visual display 400 can include two distinct paired probe regions, each of which corresponds with a pairing of separate ports 114a-d of the generator 101. In the illustrated circumstance, four RF probes (e.g., the probes 135a-d) are coupled with each of the four ports 114a-d. The first paired probe region indicates that probes 1 and 2, which correspond to the ports 114a and 114b, respectively, are paired together. The second paired probe region indicates that probes 3 and 4, which correspond to the ports 114c and 114d, respectively, are paired together.

As previously discussed, it may be desirable to achieve other pairings of the probes 1, 2, 3, 4, by which lesions may be created between the paired probes. In the illustrated embodiment, a two-way toggle virtual button 405, which is shown as a circle with substantially oppositely directed arrows inside, is provided to allow re-pairing of the probes. Pressing this toggle button 405 causes the control unit 109 to instead pair the probes 2 and 3 together and to pair the probes 1 and 4 together, as shown in FIG. 6B. Pressing the toggle button again will reassign the pairings to their original state, thus yielding once again the visual display of FIG. 6A. It may alternatively be stated that the pairing and re-pairings are made with respect to the respective ports of the generator 101.

In the illustrated embodiment, the toggle button 405 in FIG. 6B is visually distinct from the toggle button 405 as it appears in FIG. 6A. This different in appearance can assist in indicating to a user that different pairings are present in the different operational states represented in FIGS. 6A and 6B.

In some embodiments, the toggle button 405 may, for example, be a three-way toggle. For example, in certain embodiments, the 1-2 and 3-4 pairings may be the default pairing as previously discussed; pressing the button once may yield the 2-3 and 1-4 pairings as previously discussed; and pressing the button a second time may yield a 1-3 pairing and a 2-4 pairing. Pressing the button a third time may take the pairing back to the default 1-2 and 3-4 pairing configuration. Other selection buttons and mechanisms to achieve the desired pairings are contemplated. For example, rather than having a single three-way toggle button, two separate pairing buttons may be provided. In some embodiments, each pairing button includes text thereon to identify to a user the pairings that will result from pressing the buttons, such as, for example, the text “2-3; 1-4” on one button and “1-3; 2-4” on the other when the default “1-2; 3-4” pairing is present. Upon pressing either button, the selected button may repopulate with an appropriate indicator, such the text “1-2; 3-4,” to permit the user to return to the default pairing, if desired.

With reference to FIGS. 6A and 6B collectively, each paired probe region includes virtual buttons that provide temperature and lesion time information. Pressing on these virtual buttons permits alteration of these settings in manners such as discussed previously in the monopolar context.

The visual display 400 includes toggle virtual buttons to allow selection of sensory and motor testing. In FIGS. 6A and 6B, each of the sensory and motor buttons is unselected. Accordingly, a user may proceed directly to lesioning, as noted on the bottom righthand virtual button.

FIG. 6C represents the visual display that may be shown when at least onf of the sensory and motor buttons has been selected in the visual display 400 of FIG. 6A, where the default “1-2; 3-4” pairing is present. As shown in FIG. 6C, when the sensory and/or motor buttons are selected, the user is no longer provided with the option to proceed directly to lesioning, but is instead allowed to select the same bottom righthand virtual button to instead proceed to the selected stimulation event or events. That is, the bottom righthand virtual button now allows for proceeding to stimulation, rather than proceeding to lesioning in manners such as discussed previously in the monopolar context.

FIG. 6D depicts a popup display 420 that can be accessed in manners such as previously described in order to adjust specific lesion settings. In particular, temperature and lesion time can be adjusted via actuation of the temperature and lesion time buttons, in conjunction with use of the virtual number pad.

FIG. 6E depicts a visual display that is shown upon pressing the “Stimulation” button of FIG. 6C. Various options are provided to the user by way of further virtual buttons. As noted in the upper title bar, “BIPOLAR SENSORY STIMULATION” is the first available stimulation event. The user can select the appropriate buttons to start the sensory stimulation for each set of paired probes. Max voltage and ramp time may be altered in manners such as previously discussed. From this screen, the user has the option to either move on to further stimulation—that is, to perform “Motor Testing” by pressing the button thusly labeled—or to proceed directly to lesioning by pressing the “Lesion” button.

As shown above the paired probe regions of the display, the default setting is an automatic ramping of voltage for the sensory stimulation test. In some embodiments, mere turning of the dial 107 provides sufficient information or instruction to the control unit 109 (FIG. 3A) for the control unit 109 to transition voltage ramping from an automated mode to a manual mode. In the manual mode, the “Manual Ramp” title will be highlighted and underlined on the visual display, rather than the “Auto Ramp” title (which is underlined and highlighted in FIG. 6E due to the generator 101 being in the automatic ramping mode). In the alternative embodiment depicted in FIG. 6N, the bottom lefthand corner of the visual display can include a prompt to inform a user of the ability to automatically transition the ramping to manual control by turning the dial 107 (FIG. 1).

FIG. 6E depicts the visual display after the “start” button below the “1-2” probe pairing is pressed in FIG. 6D. The bipolar sensory stimulation is under way.

FIG. 6F depicts the visual display 430 after the “1-2” bipolar sensory stimulation has commenced. What was previously a “Start” button is now labeled “Stop” to provide the user with the option to pause or terminate the sensory stimulation event.

FIG. 6G depicts a visual display 440 that is shown upon pressing the “Motor Testing” button of FIG. 6E. Various options are provided to the user by way of further virtual buttons. As noted in the upper title bar, “BIPOLAR MOTOR STIMULATION” is the stimulation event at hand. The user can select the appropriate buttons to start the motor stimulation for each probe pairing. As with the sensory stimulation, maximum voltage and ramp time may be altered via the appropriate buttons. From this screen, the user can move on to a lesioning mode.

As with sensory stimulation, and as shown above the paired probe regions of the display, the default setting is an automatic ramping of voltage for the motor stimulation test. As described elsewhere herein, in some embodiments, a user can automatically transition the ramping to manual control by turning the dial 107 (FIG. 1). Stated otherwise, mere turning of the dial 107 provides information or instruction to the control unit 109 (FIG. 3) for the control unit 109 to transition voltage ramping from an automated mode to a manual mode. In the manual mode, the “Manual Ramp” title will be highlighted and underlined on the visual display, rather than the “Auto Ramp” title (which is underlined and highlighted in FIG. 6G due to the generator 101 being in the automatic ramping mode).

FIG. 6H depicts a visual display 450 that is shown upon pressing the “Lesion” button of, e.g., FIG. 6G. Various options are provided to the user by way of further virtual buttons. Bipolar lesioning via each pairing of RF probes (e.g., 135a, b or 135c, d) may be started for each pair, separately, or lesioning may be started for both pairings simultaneously, via the respective buttons that resemble similar buttons previously discussed.

In manners such as previously discussed, as a safety measure, pressing a “Start” button or the “Start All” button may not immediately transition the system to an ablation state. Instead, the “Start” or “Start All” button, upon being pressed, can be updated with a “Confirm” button to ensure that the user truly intends to start lesioning with each or both pairings of ports/devices.

FIG. 6I depicts the visual display 450 in a state in which lesioning via the first pairing has been interrupted (e.g., by a user pressing the “Stop” button) and lesioning via the second pairing is ongoing. For the first pairing, a user has the option to resume the lesioning by pressing the “Resume” button. Real-time graphical displays of temperature measurements obtained at the distal end of at least one probe of each pairing is shown beneath the “Lesion Time” region of the pairing objects.

FIG. 6J depicts a popup display 470 that can cover a portion of the display 450. In the illustrated state, lesioning via the first pairing of the ports 1 and 2 has resumed and is ongoing. The display 470 provides a detailed view of the ongoing bipolar procedure associated with the first and second ports of the generator 101. The popup display 470 can be summoned by pressing on the large virtual button associated with the first pairing FIG. 6I as the procedure is ongoing. The display 470 can be dismissed by pressing on the “X” button at the top right hand side of the display 470.

With continued reference to FIG. 6J, it is noted that the popup display 470 does not cover the homing object 230. This is also true of other popup displays. In some embodiments, the homing object 230 is shown continuously or substantially continuously throughout a selected procedure, and a user can return to the home screen 202 at any time during the procedure.

As shown in FIG. 6K, upon completion of the lesioning, a “Done” button appears. The user can press this button to proceed to display 480 of FIG. 6L, which provides a summary of the procedure. At this screen, the user is provided with the options to export data to a USB, continue lesioning, or exit via buttons provided at the bottom of the screen. In some instances, actuating the “Exit” button can return the user to the home screen 202 of FIG. 4.

Further, as previously noted, if at any point a user desires to operate the generator 101 in a different mode, the user can press the home button at the top left corner to return to the home screen of FIG. 4.

FIG. 6M depicts a popup display 490 that can cover a portion of the display 480. The display 490 provides a more detailed view of the completed monopolar procedure associated with the first port of the generator 101. The popup display 490 can be summoned by pressing on the virtual button associated with the channel 1 and channel 2 pairing of the display 480, as shown in FIG. 6L.

As previously discussed, FIG. 6N depicts an alternative embodiment of a bipolar sensory stimulation screen 431, which includes in indication 495 that alerts a user to the ability to easily transition to a manual ramp mode.

FIGS. 7A-7L depict a series of visual displays that correspond with the combination mode of an embodiment of the generator 101. Some or all of the visual displays can be displayed and/or interacted with in one or more steps of illustrative methods of an RF procedure. In some instances, the visual displays may be shown and/or viewed in the A-to-L sequence depicted.

As shown in FIG. 7A, the visual display 500 can permit probe pairing of either the probes 1 and 2 or the probes 2 and 3, but not the probes 3 and 4 because the 4 probe has not been connected. Pairings can be achieved by pressing on the “PAIR” button situated between the probes for which pairing is desired. In some embodiments a “PAIR” button (e.g., an additional or fourth “PAIR” button) will be present between the 3 and 4 probes when the 4 probe is connected. In still further embodiments, an additional “PAIR” button may also appear that permits pairing of the 1 and 4 probes. As previously discussed, pairing of probes may alternatively be termed as a pairing of ports of the generator 101.

FIG. 7B depicts a visual display after the right “PAIR” button has been selected to pair the 2 and 3 probes together for bipolar lesioning. The 1 probe will operate in monopolar mode in this configuration. The 4 probe has not been connected to a port at this stage and will not be used for lesioning in this particular setup.

FIG. 7C depicts the visual display 500 at a different starting point at which probes have been connected to all four ports. In the illustrated embodiment, three “PAIR” buttons are present between the 1, 2; 2, 3; and 3, 4 probes.

FIG. 7D depicts the visual display 500 after the 3, 4 probes/ports have been paired together.

FIG. 7E depicts a popup display 520 that can be accessed by pressing on any of the probe or probe pairing actuatable objects of FIG. 7D. The popup display 520 permits a user to select temperature and lesion time settings for each of the monopolar and bipolar components of the setup.

FIG. 7F depicts a visual display 530 that is shown upon pressing the “Stimulation” button of FIG. 7D. Each probe or probe pairing may be tested.

FIG. 7G depicts a visual display 540 that is shown upon pressing the “Motor Testing” button of FIG. 7D. Each probe or pairing may be tested.

FIG. 7H depicts a visual display 550 by which a user may proceed to conduct a combination lesion. As with other embodiments discussed above in the context of monopolar and bipolar lesions, the combination lesion can include separate “Start” buttons for each individual probe or paired set of probes. Further, a “Start All” button permits the starting of all lesions. As with other embodiments, the starting may be staggered (according to presets, which may be selected via advanced setting menus) or simultaneous. Further, pressing one of the start/start all buttons can bring up a “confirm” button for safety purposes, as previously discussed.

In FIG. 7I, a different setup is shown in which the 1 and 4 probes are monopolar and the 2 and 3 probes are paired for bipolar ablation. The 1 probe ablation has begun and been interrupted, while the 2 and 3 probe bipolar ablation and the 4 probe monopolar ablation are under way.

FIG. 7J is a popup screen associated with the number 4 probe monopolar ablation, which provides a more detailed view of the gathered data as the procedure progresses.

In FIG. 7K, all lesions are complete and the user can select the “Done” button to obtain a summary report of the lesioning activity.

FIG. 7L depicts a screen 580 that provide a combination lesion summary, similar to those described above in the monopolar and bipolar contexts.

FIGS. 8A-8C depict illustrative visual displays that correspond with portions of an illustrative pulsed (e.g., pulsed monopolar) mode of an embodiment of the generator 101. Various screens associated with the pulsed monopolar mode of operation can be similar to those described with respect to the monopolar, bipolar, and combination procedures. With respect to FIG. 8A, the screen 600 allows for a user to select settings for the pulsed monopolar procedure. Actuation of any of the probe objects in FIG. 8A causes the popup screen 620 depicted in FIG. 8B to appear. In this arrangement, a timed pulsed procedure is contemplated, so the desired temperature and lesion time for the procedure may be selected.

In FIG. 8C, however, a different type of pulsed procedure is contemplated. In this instance, a dosed pulsed monopolar procedure has been selected, e.g., via advanced settings. Accordingly, actuation of any of the probe objects in FIG. 8A causes the popup screen 620 depicted in FIG. 8C to appear. In this arrangement, the desired temperature and dose count may be selected.

In FIG. 9, a save procedures screen 700 is accessed via the home screen 202, in manners such as previously discussed. This screen allows a user to store procedure preferences. The user may select any of a variety of procedures (e.g., monopolar, bipolar, combination, pulsed monopolar, pulsed bipolar) and program in his or her preferences for such procedures. Once a particular procedure has been saved, that procedure may be recovered via this interface. Thereafter, the procedure can proceed in much the same manner as previously discussed in the various examples above.

FIG. 10 depicts one of the system settings screens 800 that may be accessible from the home screen 202—in particular, by actuating the settings object 220 (see FIG. 4). In the illustrated embodiment, the generator 101 is configurable such that a user can select preferred sound and display settings, access device information, upgrade software, set date and time preferences, select language preferences, and/or enter a demonstration mode. The sound and display preferences for the illustrated embodiment are shown on screen 800.

FIGS. 11A and 11B depict a screen 900 that permits a user to access advanced settings for the monopolar mode of operation. This screen 900 may be accessed, for example, by actuating the “Settings” button 318 on screen 300 (see FIG. 5A). A user may, for example, prefer that if both motor and sensory stimulation are to take place, that the motor stimulation precede the sensory stimulation. By default, certain embodiments conduct sensory stimulation prior to motor stimulation. The user may reverse this order by actuating the object 905. Thus, for the illustrated embodiment, the default order of operations is depicted in FIG. 11A, whereas FIG. 11B illustrates the order of operations as they will take place after the object 905 has been actuated. The object 905 may be a virtual toggle button such as those discussed elsewhere herein.

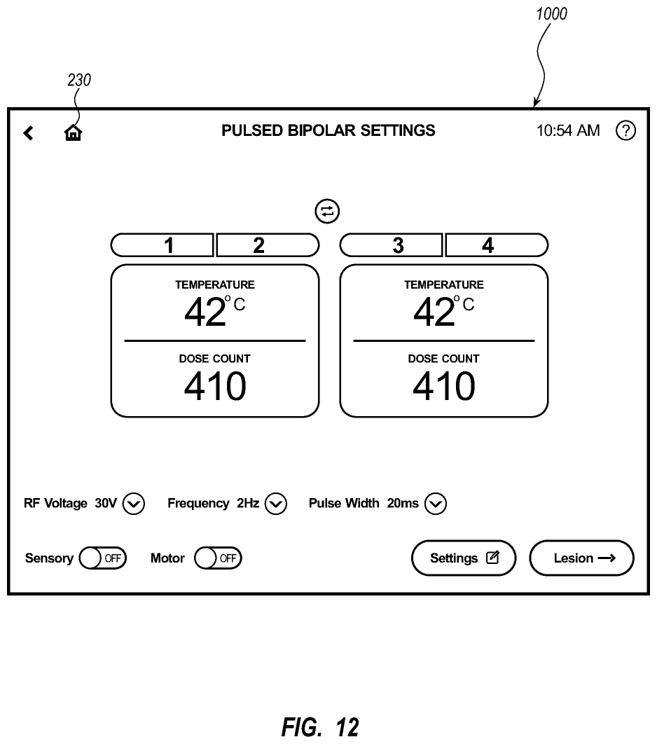

FIG. 12 depicts a pulsed bipolar setting screen 1000. In some instances, pulsed bipolar may be a less utilized procedure, and thus may not be provided as a dedicated option on the home screen 202 (FIG. 4). However, a user may be able to program preferred bipolar settings and store these as a saved procedure, which would be accessible by selecting the “save procedure” object on the home screen.

FIG. 13A depicts a flow chart 1100 showing interrelations among steps in numerous illustrative methods that may be implemented via embodiments of the system 100 and/or embodiments of the generator 101. In some embodiments, the flow chart identifies various displays that may be shown on the touchscreen 103 and the order in which those screens may be shown. FIG. 13A corresponds particularly well with embodiments of the generator 101 discussed above with respect to FIGS. 4-12. For example, the home screen 1102 can correspond with the home screen 202 described above, from which a user may select to proceed with a monopolar procedure (e.g., FIGS. 5A-5M), a bipolar procedure (e.g., FIGS. 6A-6M), a combination procedure (e.g., FIGS. 7A-7L), a pulsed monopolar procedure (e.g., FIGS. 8A-8C), or a saved procedure. Note that each such procedure corresponds with a different column in FIG. 13A. The rows of each such column, however, are generally the same. Stated otherwise, the generator 101 can be configured to step a user through any of the selected procedures in a predictable and, over time, familiar manner.

FIG. 13B depicts another flow chart 1200 showing interrelations among steps in numerous illustrative methods that may be implemented via embodiments of the system 100 and/or embodiments of the generator 101. In this embodiment, the home screen 1202 can be substantially the same as the home screens 202, 1202 described above. However, in this instance, the order in which motor and sensory stimulation takes place is reversed from that of flow chart 1100. In some instances, such a reversal within the preset procedural flow can be accomplished via actuation of the object depicted in FIG. 11A and FIG. 11B, as discussed above.

FIG. 13C depicts another flow 1300 chart showing interrelations among steps in numerous illustrative methods that may be implemented via embodiments of the system 100 and/or embodiments of the generator 101. In this embodiment, a home screen 1302 associated with the generator 101 includes an actuatable object for directly accessing a pulsed bipolar procedure, and generator 101 is configured to step a user through such a procedure in manners such as those described previously with respect to other procedures (e.g., monopolar, bipolar, combination, pulsed monopolar).

It is noted that one or more of the functions achievable by virtual buttons provided to the touchscreen may additionally or alternatively be implemented by dedicated physical buttons located on the generator 101. Moreover, actions disclosed herein may described as methods. Stated otherwise, the present disclosure and claims that follow provide support for various methods of operating systems, generators, or control units, of performing RF procedures, of activating probes, of activating RF needles, etc.

Any suitable combination of the various features of the various embodiments disclosed herein is contemplated. Moreover, any suitable combination of the various features of the various embodiments disclosed herein and those of the aforementioned U.S. patent applications are also contemplated.

The term “coupled to” can mean connected to in any suitable fashion, whether that coupling is direct or indirect, wired or wireless, etc. Separate components may be coupled to each other. Moreover, in some instances, where separately identified components are integrally formed from a unitary piece of material, or stated otherwise, are included together in a monolithic element, those elements may also be said to be coupled to one another.

Although the foregoing detailed description contains many specifics for the purpose of illustration, a person of ordinary skill in the art will appreciate that many variations and alterations to the following details can be made and are considered to be included herein. Accordingly, the foregoing embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description. These additional embodiments are determined by replacing the dependency of a given dependent claim with the phrase “any one of claims [x] through the immediately preceding claim,” where the bracketed term [x] is replaced with the number of the most recently recited independent claim. For example, for the first claim set that begins with independent claim 1, claim 3 can depend from either of claims 1 and 2, with these separate dependencies yielding two distinct embodiments; claim 4 can depend from any one of claim 1, 2, or 3, with these separate dependencies yielding three distinct embodiments; claim 5 can depend from any one of claim 1, 2, 3, or 4, with these separate dependencies yielding four distinct embodiments; and so on.

Recitation in the claims of the term “first” with respect to a feature or element does not necessarily imply the existence of a second or additional such feature or element. Elements specifically recited in means-plus-function format, if any, are intended to be construed in accordance with 35 U.S.C. § 112(f). Elements not presented in requisite means-plus-function format are not intended to be construed in accordance with 35 U.S.C. § 112(f). Embodiments of the invention in which an exclusive property or privilege is claimed are defined as follows. application:

1. A generator comprising:
a plurality of ports, each port being configured to individually couple with a separate radiofrequency probe;
a touchscreen; and
a control unit configured to control delivery of radiofrequency energy to the plurality of ports to operate the generator in each of a monopolar mode and a bipolar mode, the control unit being communicatively coupled with the touchscreen and configured to:
cause the touchscreen to show a home display that includes a monopolar object and a bipolar object;
operate the plurality of ports in the monopolar mode upon receiving a control signal from the touchscreen corresponding with actuation of the monopolar object or operate at least two of the plurality of ports as a coupled bipolar pair in the bipolar mode upon receiving a control signal from the touchscreen corresponding with actuation of the bipolar object;
cause the touchscreen to continuously display a homing object while the generator is in either the monopolar mode or the bipolar mode; and
upon actuation of the homing object, cause the touchscreen to show the home display again.

2. The generator of claim 1, wherein upon the touchscreen showing the home display again, each of the monopolar object and the bipolar object is actuatable to either enter the monopolar mode or the bipolar mode, respectively.

3. The generator of claim 1, wherein the home display further includes a combination object that is selectable to cause the generator to operate in a combination mode in which a pair of the ports are coupled together in a bipolar arrangement and at least one further port is operable in a monopolar mode.

4. The generator of claim 1, wherein the home display further includes a pulsed monopolar object that is selectable to cause the generator to operate in a pulsed monopolar mode in which at least one of the ports delivers pulsed radiofrequency energy.

5. The generator of claim 1, wherein when either the monopolar mode or the bipolar mode is selected and entered into, the control unit causes the touchscreen to show at least one display dedicated to the selected mode via which settings of the selected mode are selectable and/or confirmable prior to showing a further display that includes one or more objects that are actuatable to energize one or more of the ports for ablation in the selected mode.

6. The generator of claim 5, wherein said at least one display dedicated to the selected mode includes one or more objects that are actuatable to cause the control unit to show, prior to showing the further display for ablation in the selected mode, one or more of:
a sensory stimulation display that includes one or more objects actuatable to perform one or more sensory stimulation events via one or more of the ports; and
a motor stimulation display that includes one or more objects actuatable to perform one or more motor stimulation events via one or more of the ports.

7. A generator comprising:
at least three ports, each port being configured to individually couple with a separate radiofrequency probe;
a touchscreen; and
a control unit configured to control delivery of radiofrequency energy to the ports, the control unit being communicatively coupled with the touchscreen,
wherein the generator is configured to operate in each of a monopolar mode in which at least one of the ports is configured for monopolar ablation, a bipolar mode in which at least two of the ports are paired together for bipolar ablation, and a combination mode in which two of the three ports are paired together for bipolar ablation and the remaining one of the three ports is configured for monopolar ablation, and
wherein the control unit is configured to cause the touchscreen to show a home display that comprises a first object via which the monopolar mode is selectable, a second object via which the bipolar mode is selectable, and a third object via which the combination mode is selectable.

8. The generator of claim 7, wherein a combined area of the first, second, and third objects collectively accounts for no less than one fifth of the displayable area of the touchscreen.

9. The generator of claim 8, wherein the combined area of the first, second, and third virtual buttons collectively accounts for no less than one fourth of the displayable area of the touchscreen.

10. The generator of claim 7, wherein each of the first and second objects is bigger than the third object.

11. The generator of claim 7, wherein the first and second objects are the same size as each other.

12. The generator of claim 7, wherein settings of the monopolar mode, the bipolar mode, and the combination mode are not directly editable on the home display.

13. The generator of claim 7, wherein at least one of the first object, the second object, the third object, or one or more additional objects must be actuated on the home display to cause at least one additional display to be shown on the touchscreen before edits to settings of any of the monopolar mode, the bipolar mode, or the combination mode can be made.

14. The generator of claim 7, wherein the home display is devoid of visual indicia specifically associated or associable with any of the at least three ports.

15. The generator of claim 7, wherein the home display further comprises a pulsed monopolar object via which a pulsed monopolar mode of the generator is selectable.

16. The generator of claim 7, wherein the home display further comprises a saved procedure object via which one or more further operational modes of the generator can be accessed.

17. The generator of claim 7, wherein no further objects are present on the home display via which operational modes of the generator are accessible.

18. The generator of claim 7, wherein a settings object via which advanced settings of the generator are accessible is the only other actuatable object present on the home display.

19. The generator of claim 7, wherein a settings object via which advanced settings of the generator are accessible and a help object via which help information regarding the generator are accessible are the only other actuatable objects present on the home display.

20. The generator of claim 7, wherein once the generator has entered one of the monopolar, bipolar, or combination modes, a homing object must be actuated to cause the home display to be shown again on the touchscreen in order to enter either of the remaining monopolar mode, bipolar mode, or combination mode.

21. The generator of claim 20, wherein the homing object is continuously displayed on the touchscreen throughout operation of the generator in each of the monopolar mode, bipolar mode, and combination mode.

22. The generator of claim 20, wherein said entering either of the remaining monopolar mode, bipolar mode, or combination mode is achieved by selecting the first object, the second object, or the third object, respectively, on the home display.

23-41. (canceled)

42. A method of controlling delivery of radiofrequency energy via a generator, the generator comprising a plurality of ports, each port being configurable to be coupled with separate radiofrequency probes, the method comprising:
causing a touchscreen to show a home display that includes a monopolar object and a bipolar object;
operating:
the plurality of ports in a monopolar mode upon receiving a control signal from the touchscreen corresponding with actuation of the monopolar object; or
at least two of the plurality of ports as a coupled bipolar pair in a bipolar mode upon receiving a control signal from the touchscreen corresponding with actuation of the bipolar object;
causing the touchscreen to continuously display a homing object while the generator is in either the monopolar mode or the bipolar mode; and
upon actuation of the homing object, causing the touchscreen to show the home display again.

43. (canceled)
